(19) United States
(12) Patent Application Publication
(10) Pub. No.: US 2024/0359175 A1
(43) Pub. Date: Oct. 31, 2024

(54) MICROFLUIDIC DEVICES FOR ENHANCED MICROMIXING AND DETECTION SAMPLING

(71) Applicant: BATTELLE SAVANNAH RIVER ALLIANCE, LLC, AIKEN, SC (US)

(72) Inventors: CHRISTOPHER A. SOBECKI, AIKEN, SC (US); STEVEN M. DEMERS, NORTH AUGUSTA, SC (US)

(21) Appl. No.: 18/641,918

(22) Filed: Apr. 22, 2024

Related U.S. Application Data
(60) Provisional application No. 63/497,787, filed on Apr. 24, 2023.

(51) Int. Cl. B01L 3/00; B01J 19/00; B82Y 15/00; G01N 21/25; G01N 21/27

(52) U.S. Cl. CPC B01L 3/502715; B01J 19/0093; B01L 3/502761; B82Y 15/00; G01N 21/27; G01N 2021/258

(57) ABSTRACT

A device, system, and method incorporating a microfluidic device for enhanced mixing and detection sampling is disclosed. The microfluidic device may include one or more streams comprising a sensing solution and an analyte solution. The sensing solution and the analyte solution may be mixed in the microfluidic device and may be used to determine whether the analyte solution contains one or more chemical, biological, and/or radiological/nuclear (CBRN) threats.

### RELATED APPLICATIONS

The present application is based on and claims priority to U.S. Provisional Patent Application Ser. No. 63/497,787, filed on Apr. 24, 2023, which is incorporated herein by reference in its entirety.

### FEDERAL RESEARCH STATEMENT

This invention was made with government support under Contract No. 89303321CEM000080, awarded by the U.S. Department of Energy. The government has certain rights in the invention.

### SEQUENCE LISTING

The instant application contains a Sequence Listing which has been submitted electronically in XML file format and is hereby incorporated by reference in its entirety. Said XML copy, created on Jun. 4, 2024, is named BSRA-318_SRS-021-014_SL.xml and is 5,781 bytes in size.

### FIELD

This present subject matter relates generally to the mixing and detecting chemical, biological and/or radiological/nuclear (CBRN) threats, more particularly, to microfluidic devices for the mixing and detecting chemical, biological, and/or radiological/nuclear (CBRN) threats.

### BACKGROUND

Due to the risk of chemical, biological, and radiological/nuclear (CBRN) threats, groups such as emergency responders, law enforcement, military personnel, and firefighters prioritize the fast and efficient analysis of CBRN threats. However, traditional devices utilized to test and analyze samples, such as environmental samples, for CBRN threats may have limited efficacy, may waste valuable time in analyzing the samples, and may even provide an excessive number of false positives or false negatives.

Notably, advanced sensor capabilities for onsite detection of chemical, biological, and radiological/nuclear (CBRN) threats are important to significantly limit the risk of exposure to persons at such sites and allow for the rapid, in-field collection of essential scientific data and critical evidence. However, current scientific and commercial sensing capabilities generally require highly specific and ultra-sensitive methods that are often energy-intensive or require offsite post-analysis for positive detection, leaving onsite personnel vulnerable. Moreover, current in-field sensors are typically limited to detecting only one class of threat at a time.

Current detection techniques for CBRN are generally complex and require highly specific and ultra-sensitive methods. For example, biological threats, such as anthrax or ricin, are routinely analyzed offsite through a laboratory response network, where samples of interest are first sent to a sentinel lab, followed by a reference laboratory, and then, if the biological threats are not ruled out at the first two laboratories, the samples are sent to a national laboratory. This network uses fixed laboratory equipment, such as mass spectroscopy (MS) techniques, which are highly sensitive (ricin reported detection limits of 0.64 ng/ml in the literature). Unfortunately, they require complex data analysis, especially for biological samples, which have numerous MS fragments and potential matrix effects that can interfere with the determination of the analyte.

Accordingly, there is a need for enhanced sensing technology, particularly a sensing technology that allows for a low detection time.

### SUMMARY OF THE INVENTION

Aspects and advantages of the invention will be set forth in part in the following description, or may be obvious from the description, or may be learned through practice of the invention.

In one aspect, the present subject matter is directed to a system for mixing and detecting chemical, biological and/or radiological/nuclear (CBRN) threats, the system comprising: a microfluidic device, the microfluidic device comprising one or more inlet channels and one or more outlet channels; a reservoir, the reservoir being in fluid communication with the microfluidic device; and a sensing solution, the sensing solution comprising a sensor for the detection of one or more chemical, biological and/or radiological/nuclear (CBRN) threats, the sensor comprising: a nanoparticle; and a first energy transfer pair comprising a first donor, a first acceptor, and a first linking agent linking the first donor and the first acceptor, the linking agent comprising a first polynucleic acid, the first polynucleic acid comprising a first specific binding ligand for a first CBRN threat, wherein the nanoparticle is the first donor or the first acceptor.

In another aspect, the present subject matter is directed to a system for mixing and detecting chemical, biological and/or radiological/nuclear (CBRN) threats, the system comprising: a microfluidic device, the microfluidic device comprising two or more inlet channels and one or more outlet channels, the two or more inlet channels comprising a sensing solution and an analyte solution, the sensing solution comprising a sensor for the detection of one or more chemical, biological and/or radiological/nuclear (CBRN) threats; and a reservoir, the reservoir being in fluid communication with the microfluidic device.

In yet another aspect, the present subject matter is directed to a system for mixing and detecting chemical, biological and/or radiological/nuclear (CBRN) threats, the system comprising: a mobile analysis device, the mobile analysis device comprising: a microfluidic device, the microfluidic device comprising a sensor for the detection of one or more chemical, biological and/or radiological/nuclear (CBRN) threats; and a reservoir, the reservoir being in fluid communication with the microfluidic device.

In one aspect, the present subject matter is directed to a method for mixing and detecting chemical, biological and/or radiological/nuclear (CBRN) threats, the method comprising: contacting a sensing solution with an analyte solution in a microfluidic device, the sensing solution comprising a sensor, the sensor comprising a nanoparticle; a first energy transfer pair comprising a first donor, a first acceptor, and a first linking agent linking the first donor and the first acceptor, the first linking agent comprising a first polynucleic acid that includes a first specific binding ligand for a first CBRN threat; and examining the sensor for an optically detectable signal after contact of the sensing solution and the analyte solution.

### BRIEF DESCRIPTION OF THE FIGURES

A full and enabling disclosure of the present subject matter, including the best mode thereof, directed to one of ordinary skill in the art, is set forth in the specification, which makes reference to the appended figures, in which:

FIG. 1 schematically illustrates one embodiment of a single energy transfer pair of a sensor as disclosed herein;

FIG. 2 schematically illustrates another embodiment of a single energy transfer pair of a sensor as disclosed herein;

FIG. 3 schematically illustrates a signaling mechanism of a single energy transfer pair of a sensor as described herein;

FIG. 4 schematically illustrates one embodiment of a multi-functional CBRN sensor as described herein;

FIG. 5 schematically illustrates the working principal of one embodiment of a multi-functional CBRN sensor as described herein;

FIG. 6 schematically illustrates one embodiment of a sensing system encompassed herein;

FIG. 7 schematically illustrates another embodiment of a sensing system encompassed herein;

FIG. 8 schematically illustrates one embodiment of a sampling medium as may be utilized in a sensing system as described herein;

FIG. 9A illustrates the calculated Forster distances for both FRET and NSET interactions between a metal nanoparticle (gold, silver, or copper) and a dye as may be utilized in sensors as described herein;

FIG. 9B illustrates the calculated normalized extinction spectrum of different metal nanoparticles and the overlay of that of the exemplary dye molecule (tetrachlorofluorescein (TET)) of the sensors of FIG. 9A;

FIG. 9C illustrates the calculated quenching efficiency of different metal nanoparticles of the sensors of FIG. 9A;

FIG. 10 illustrates the UV-vis absorbance of plain gold nanoparticles and gold nanoparticles conjugated to DNA as may be utilized in disclosed sensors;

FIG. 11A illustrates the spectral overlap of a representative donor and acceptor as may be utilized in disclosed sensors;

FIG. 11B illustrates the decrease in quantum dot photoluminescence (PL) after binding the donor of FIG. 11A to the quenching acceptor;

FIG. 12 illustrates the spectral overlap between a quantum dot donor and a quantum dot acceptor as may be utilized in disclosed sensors;

FIG. 13 illustrates the three-dimensional fluorescence excitation emission matrix (EEM) spectroscopy results for the quantum dot pair of FIG. 12 in the absence of FRET;

FIG. 14A illustrates the normalized UV-Vis absorbance of quantum dots of different sizes as may be utilized in disclosed sensors;

FIG. 14B illustrates the emission intensity of quantum dots of different sizes as may be utilized in disclosed sensors;

FIG. 15 illustrates investigative results of one embodiment of a sensor as described herein;

FIG. 16A illustrates a portion of the spectral overlap of FIG. 11A in more detail;

FIG. 16B provides results of sensitivity measurements for a sensor including the donor and acceptor of FIG. 16A and including an analyte targeting DNA spacer between the FRET components as compared to a similar system with no added spacer between the FRET components;

FIG. 17 illustrates the predicted secondary structure of SEQ ID NO: 6;

FIG. 18A illustrates absorbance spectra of various combinations of components of a sensor as disclosed herein;

FIG. 18B illustrates emission spectra of a sensor and components of the sensor of FIG. 18A;

FIG. 18C illustrates fluorescence ratios of the sensor and components of the sensor of FIG. 18A;

FIG. 19 presents results upon selective binding of potential specific binding agents for strontium (Sr) followed by sorting using BD FACSMelody™;

FIG. 20 illustrates a perspective view of one embodiment of a microfluidic device in accordance with aspects of the present subject matter;

FIG. 21 illustrates a perspective view of another embodiment of a microfluidic device in accordance with aspects of the present subject matter;

FIG. 22 illustrates a perspective view of one embodiment of a system in accordance with aspects of the present subject matter;

FIG. 23 illustrates a perspective view of another embodiment of a system in accordance with aspects of the present subject matter; and

FIG. 24 illustrates a perspective view of a further embodiment of a system in accordance with aspects of the present subject matter.

Repeat use of reference characters in the present specification and drawings is intended to represent the same or analogous features or elements of the present invention.

### DETAILED DESCRIPTION

Reference now will be made in detail to embodiments of the invention, one or more examples of which are illustrated in the drawings. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment can be used with another embodiment to yield still a further embodiment. Thus, it is intended that the present invention covers such modifications and variations as come within the scope of the appended claims and their equivalents.

The present subject matter relates to a microfluidic device, system, and method capable of the simultaneous detection of multiple chemical, biological, and radiological/nuclear (CBRN) threats (e.g., chemical agents, biological agents, radiological/nuclear agents) and, more particularly, to a microfluidic device, system, and method that may utilize multiplexed Forster Resonance Energy Transfer based (FRET-based) and/or Nanometal Surface Energy Transfer based (NSET-based) sensors capable of simultaneous detection of multiple threats, including one or more CBRN threats. Specifically, the present subject matter relates to a microfluidic device, system, and method including a multifunctional sensor that can simultaneously detect two or more different CBRN threats of the same or different classifications. For instance, two or more different CBRN threats may include two different chemical agents, two different biological agents, two different radiological/nuclear agents, or a combination thereof. The disclosed microfluidic device, system, and method can be used to simultaneously detect CBRN threats with high selectivity, high confidence, and reduced false positives. Notably, utilizing a microfluidic device and/or system formed in accordance with the present disclosure may be particularly advantageous as the mass transfer and heat transfer between two or more fluid streams may be enhanced as compared to traditional mixing apparatuses or devices.

It should be understood that throughout the entirety of this specification, each numerical value disclosed should be read as modified by the term “about”, unless already expressly so modified, and then read again as not to be so modified. For instance, a value of “100” is to be understood as disclosing “100” and “about 100”. Further, it should be understood that throughout the entirety of this specification, when a numerical range is described, any and every amount of the range, including the end points and all amounts therebetween, is disclosed. For instance, a range of “1 to 100”, is to be understood as disclosing both a range of “1 to 100 including all amounts therebetween” and a range of “about 1 to about 100 including all amounts therebetween”. The amounts therebetween may be separated by any incremental value. Notably, some aspects of the present disclosure may omit one or more of the features disclosed herein.

Generally, a microfluidic device may include one or more inlets, one or more outlets, one or more inlet channels, one or more outlet channels, one or more junctions, or a combination thereof. As used herein, the term “inlet channel” refers to a microfluidic device channel connected to a microfluidic device inlet. The one or more inlet channels may comprise one or more inlet channel streams. As used herein, the term “outlet channel” refers to a microfluidic device channel connected to a microfluidic device outlet. The one or more outlet channels may comprise one or more outlet channel streams. An inlet channel stream and/or an outlet channel stream may comprise a gas, a liquid, a solid, or a combination thereof. In general, one or more inlets, one or more outlets, one or more inlet channels, one or more outlet channels, one or more junctions, or a combination thereof of a microfluidic device may be in fluid communication. In some aspects, one or more inlets, one or more outlets, one or more inlet channels, one or more outlet channels, one or more junctions, or a combination thereof of a microfluidic device may be connected. Generally, the one or more inlet channels and/or one or more outlet channels may be connected to one or more converging junctions, one or more diverging junctions, one or more T-junctions, one or more Y-junctions, or a combination thereof.

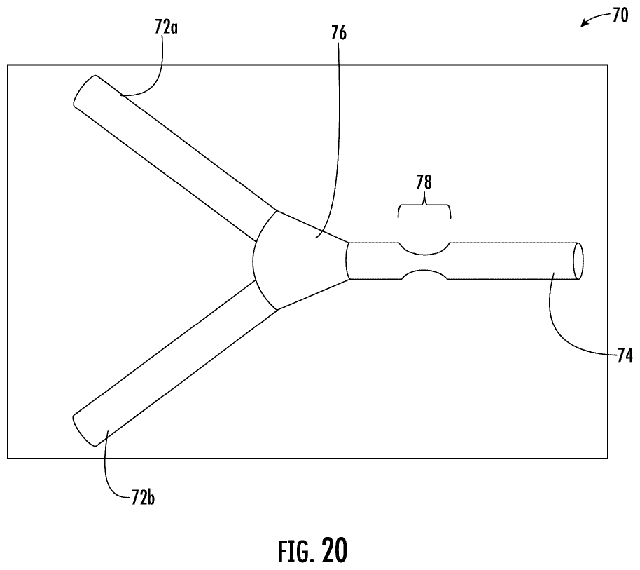

Generally, the microfluidic device may have any shape (e.g., cylindrical, rectangular) and/or size. For instance, FIG. 20 illustrates a rectangular microfluidic device 70. The microfluidic device may be two-dimensional or three-dimensional.

Generally, one or more channels of a microfluidic device may be configured to mix or contact two or more streams (e.g., two or more inlet channel streams). In this respect, two or more streams may combine in a microfluidic device such that they form a mixture. For instance, a microfluidic device may be configured to mix and/or contact a sensing solution and an analyte solution. In this respect, a first inlet channel stream containing a sensing solution and a second inlet channel stream containing an analyte solution may mix or come into contact. In general, the sensing solution may be directed through a first inlet channel of the microfluidic device and the analyte solution may be directed through a second inlet channel of the microfluidic device before the sensing solution and the analyte solution come into contact or mix. Advantageously, the mixing of two or more streams in the microfluidic device may enhance the efficiency of the mixing. In this respect, the utilization of the microfluidic device in the mixing of two or more streams may increase the mixing rate and efficiency of the two or more streams, decrease the detection time of one or more analytes, and/or lower the detection limit. Generally, one or more streams of the microfluidic device may have laminar flow, turbulent flow, or a combination thereof.

In some aspects, one or more streams may have solid particles dispersed therein. In some aspects, one or more streams may move through one or more inlet channels and/or one or more outlet channels of a microfluidic device via a pressure differential and/or the pumping of one or more streams through one or more channels of a microfluidic device.

Generally, an inlet channel stream and/or an outlet channel stream of a microfluidic device in accordance with the present disclosure may comprise a sensing solution. The sensing solution may comprise one or more of any of the sensors disclosed herein. The one or more sensors of the sensing solution may have any of the sensor properties, characteristics, components, configurations, or a combination thereof as disclosed herein. Additionally, a microfluidic device, a microfluidic cartridge, or a system incorporating a microfluidic device and/or microfluidic cartridge in accordance with the present disclosure may include any of the sensing system properties, characteristics, components, configurations, or a combination thereof as disclosed herein. Notably, a device comprising a microfluidic device having at least a portion of a container or reservoir affixed and/or mounted to the microfluidic device may be referred to as a “microfluidic cartridge”. In some aspects, an inlet channel stream and/or an outlet channel stream of a microfluidic device may comprise an analyte that represents or is indicative of a CBRN threat. In this respect, an inlet channel stream and/or an outlet channel stream of the microfluidic device may comprise an analyte solution. In general, the analyte solution may comprise one or more threat agents. Notably, the analyte solution may be a sample suspected of containing one or more threat agents, which may be referred to herein as a “test sample”.

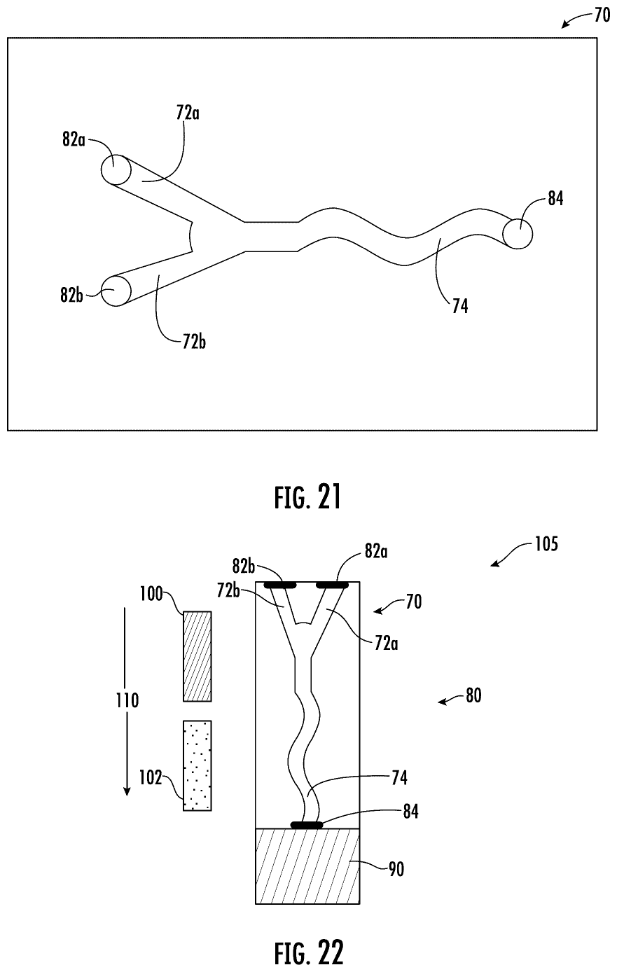

Generally, an inlet channel and/or an outlet channel may be cylindrical or rectangular. In general, an inlet channel and/or an outlet channel may be linear or non-linear. In some aspects, an inlet channel and/or an outlet channel may be a straight channel, a spiral channel, a curved channel (e.g., a serpentine channel, a sinusoidal channel), or a combination thereof. For instance, as illustrated in FIG. 20, inlet channels 72a, 72b and outlet channel 74 are straight, cylindrical channels. Notably, when an inlet channel and/or an outlet channel are cylindrical, the diameter of the respective channel may be constant or may vary. In this respect, the diameter of an inlet channel and/or an outlet channel may have one or more constriction portions that narrow the diameter of the channel. For instance, as illustrated in FIG. 20, an outlet channel 74 of a microfluidic device 70 may have a throat 78 that narrows the diameter of the outlet channel 74 to a specific throat width or throat diameter. As further illustrated in FIG. 20, a microfluidic device 70 may have two inlet channels 72a and 72b that converge at a Y-junction 76. Further, as illustrated in FIG. 21, a microfluidic device 70 may have a non-linear, sinusoidal outlet channel 74, an outlet 84, inlet channels 72a and 72b, and inlets 82a and 82b.

Generally, two or more streams of a microfluidic device may be mixed passively or actively. For instance, passive mixing may include altering the configuration or structure of an inlet channel and/or an outlet channel of the microfluidic device to mix two or more streams. Further, for instance, if two or more fluid streams are mixed actively, the mixing of the two or more fluid streams may be manipulated or influenced by a temperature gradient, a magnetic field, sound waves, electricity, or a combination thereof, which may increase the mixing rate of two or more inlet channel streams, which may contain a sensing solution and an analyte solution. The temperature gradient may be applied by a heating source (e.g., a hot plate, a heating coil, etc.) or a combination of heating sources and a cooling source (e.g., a Peltier cooling device, a cooling tube, etc.) or a combination of cooling sources. It should be understood that a heating source and/or a cooling source may be outside of a microfluidic device and/or inside a microfluidic device. Notably, a heating source and/or a cooling source may be a component of a microfluidic device or a system in accordance with the present disclosure. In general, a heating source and/or a cooling source may be positioned adjacent or relative to a microfluidic device and/or a microfluidic cartridge. In general, a heating source and/or a cooling source may use electricity or a liquid to heat or cool a microfluidic device. For instance, a heating source may heat-up via electricity and a cooling source may use a cooling fluid.

In general, when a temperature gradient is applied to a microfluidic device and/or a microfluidic cartridge, the temperature gradient may be applied such that the temperature of one or more streams and/or one or more mixtures of the microfluidic device and/or a microfluidic cartridge are cooled at a rate of about −0.1° C./min to about −50° C./min, including all increments of 0.1° C./min therebetween. Generally, the temperature of one or more streams and/or one or more mixtures of a microfluidic device and/or a microfluidic cartridge may be cooled at a rate of about −0.1° C./min or less, such as about −0.5° C./min or less, such as about −1° C./min or less, such as about −5° C./min or less, such as about −10° C./min or less, such as about −25° C./min or less. In general, the temperature of one or more streams and/or one or more mixtures of a microfluidic device and/or a microfluidic cartridge may be cooled at a rate of about −50° C./min or more, such as about −25° C./min or more, such as about −10° C./min or more, such as about −5° C./min or more, such as about −1° C./min or more, such as about −0.5° C./min or more.

Referring now to FIG. 22, FIG. 22 illustrates a system 105 comprising a microfluidic cartridge 80 comprising a microfluidic device 70 affixed to a container or reservoir 90. Notably, a sensing solution and an analyte solution may enter the microfluidic device separately at microfluidic device inlets 82a and 82b. Then, the sensing solution and the analyte solution may flow through inlet channels 72a and 72b until the inlet channels meet or converge. Next, the sensing solution and the analyte solution may contact and mix to form a mixture in outlet channel 74. Then, the mixture of the sensing solution and the analyte solution may exit the microfluidic device 70 via the microfluidic device outlet 84 and enter the reservoir 90 affixed to the microfluidic device 70. As further illustrated in FIG. 22, a temperature gradient 110 may influence the mixing of the sensing solution and the analyte solution. In this respect, a heating source 100 and a cooling source 102 may create a temperature gradient 110 in the microfluidic device 70 to influence the mixing of the sensing solution and the analyte solution. The temperature gradient 110 of FIG. 22 is configured such that the temperature decreases from the microfluidic device inlets 82a and 82b to the microfluidic device outlet 84. In general, the temperature gradient of a microfluidic device may be such that the temperature of one or more streams decreases or increases from one or more microfluidic device inlets to one or more microfluidic device outlets.

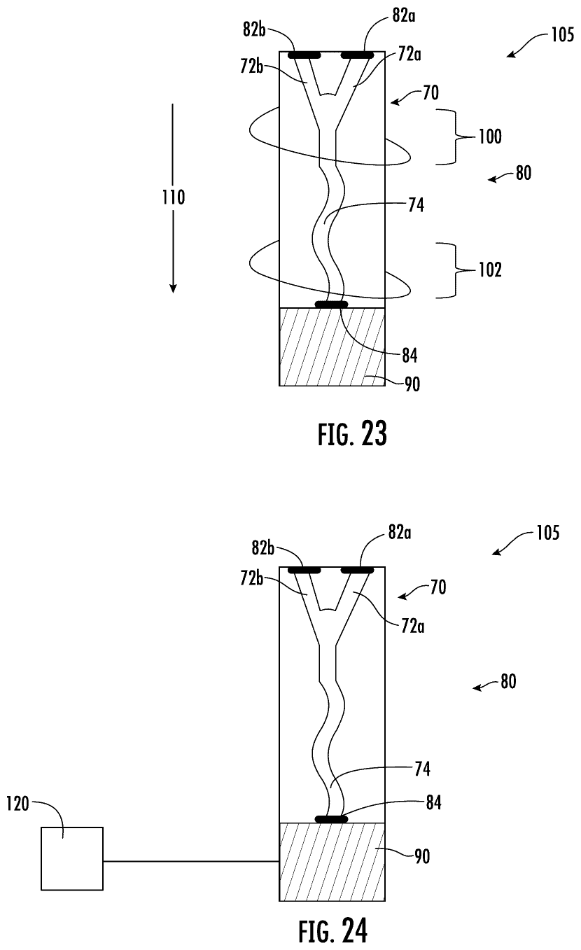

Referring now to FIG. 23, FIG. 23 illustrates a system 105 comprising a microfluidic cartridge 80 comprising a microfluidic device 70 affixed to a container or reservoir 90. Notably, a sensing solution and an analyte solution may enter the microfluidic device separately at microfluidic device inlets 82a and 82b. Then, the sensing solution and the analyte solution may flow through inlet channels 72a and 72b until the inlet channels meet or converge. Next, the sensing solution and the analyte solution may contact and mix to form a mixture in outlet channel 74. Then, the mixture of the sensing solution and the analyte solution may exit the microfluidic device 70 via the microfluidic device outlet 84 and enter the reservoir 90 affixed to the microfluidic device 70. As further illustrated in FIG. 23, a temperature gradient may influence the mixing of the sensing solution and the analyte solution. In this respect, a heating source 100, which is a coil in FIG. 23, and a cooling source 102, which is a coil in FIG. 23, may create a temperature gradient 110 in the microfluidic device 70 to influence the mixing of the sensing solution and the analyte solution. The temperature gradient 110 of FIG. 23 is configured such that the temperature decreases from the microfluidic device inlets 82a and 82b to the microfluidic device outlet 84.

In general, after two or more streams (e.g., two or more inlet channel streams) of the microfluidic device are mixed, the resulting mixture may be collected into a container or reservoir for spectrophotometric measurements and/or analysis. For instance, a mixture of a sensing solution and an analyte solution may be analyzed in a container or reservoir via spectrophotometric measurements. As previously disclosed herein, FIGS. 22 and 23 illustrate a container or reservoir 90 affixed to a microfluidic device 70. In general, the container or reservoir may be a cuvette, such as a 1 cm square cuvette. It should be understood that the container or reservoir may be any container or reservoir, such as a container or reservoir compatible with a spectrophotometric device. As illustrated in FIG. 24, a system 105 may comprise a spectrophotometric device 120 configured to analyze or measure a mixture in a container or reservoir 90. Additionally, it should be understood that a spectrophotometric device may be configured to analyze or measure a mixture in one or more outlet channels. The spectrophotometric device may be configured to obtain spectrophotometric measurements of a mixture of the sensing solution and the analyte solution. In some aspects, the container or reservoir may be in fluid communication with any of the components of a microfluidic device, such as the outlet of a microfluidic device. For instance, as illustrated in FIGS. 22 and 23, a microfluidic device 70 may have an outlet 84 that is in fluid communication with a container or reservoir 90. In some aspects, a container or reservoir may be separated from the microfluidic device except for the structure that provides for fluid communication between the microfluidic device and the container or reservoir. In this respect, a container or reservoir may not be affixed and/or mounted to a microfluidic device, but instead may be connected to a component of a microfluidic device (e.g., a microfluidic device outlet) via one or more tubes and/or pipes. In general, the structure that provides for fluid communication between the microfluidic device and the container or reservoir may be a singular tube or a system of tubes capable of carrying one or more streams, such as a mixture of a sensing solution and an analyte solution.

Generally, when at least a portion of a container or reservoir is affixed and/or mounted to a microfluidic device, the container or reservoir may be removable from the microfluidic device. In this respect, a container or reservoir may be removed from a microfluidic device after a sample, such as a mixture of a sensing solution and an analyte solution, is collected in the container or reservoir. In general, a mixture of a sensing solution and an analyte solution may be measured and/or analyzed by a spectrophotometric device before and/or after the removal of a container or reservoir from a microfluidic device. In some aspects, a mixture of a sensing solution and an analyte solution may be measured and/or analyzed by a spectrophotometric device without removing the container or reservoir from the microfluidic device. In this respect, a mixture of a sensing solution and an analyte solution collected in a container or reservoir of a microfluidic cartridge may be measured and/or analyzed by a spectrophotometric device without the removal of the container or reservoir.

Generally, following the combination of the sensors of a sensing solution with threat agents from an analyte solution, the mixture of the sensing solution and the analyte solution can be held in optical communication with a light source, e.g., via an excitation monochromator and also in optical communication with a detector, e.g., via an emission monochromator at a suitable angle to one another, e.g., 90°. The detector can provide an output, e.g., an emission spectrum, that can provide an indication of the presence of one or more of the chemical threats capable of detection by the multiplexed sensor. Any of the components mentioned above (e.g., detector) may be incorporated into a system and/or a mobile analysis device in accordance with the present disclosure.

In some aspects, as illustrated in FIGS. 22 and 23, the microfluidic device 70 and/or the microfluidic cartridge 80 may have the shape of a cuvette. Notably, as previously disclosed herein, a device comprising a microfluidic device having at least a portion of a container or reservoir affixed and/or mounted to the microfluidic device may be referred to as a “microfluidic cartridge”. For instance, the devices having reference numeral 80 illustrated in FIGS. 22 and 23 are microfluidic cartridges.

Notably, an inlet channel and/or an outlet channel of the microfluidic device may have a channel height and/or width of from about 0.5 microns to about 1 millimeter, including all increments of about 0.5 microns therebetween. For instance, an inlet channel and/or an outlet channel may have a channel height and/or width of about 0.5 microns or more, such as about 1 micron or more, such as about 5 microns or more, such as about 10 microns or more, such as about 15 microns or more, such as about 20 microns or more, such as about 25 microns or more, such as about 30 microns or more, such as about 40 microns or more, such as about 50 microns or more, such as about 100 microns or more, such as about 150 microns or more, such as about 200 microns or more, such as about 300 microns or more, such as about 400 microns or more, such as about 500 microns or more. In general, an inlet channel and/or an outlet channel may have a channel height and/or width of about 1 millimeter or less, such as about 500 microns or less, such as about 400 microns or less, such as about 300 microns or less, such as about 200 microns or less, such as about 150 microns or less, such as about 100 microns or less, such as about 50 microns or less, such as about 40 microns or less, such as about 30 microns or less, such as about 25 microns or less, such as about 20 microns or less, such as about 15 microns or less, such as about 10 microns or less. Notably, when the inlet channel and/or the outlet channel of a microfluidic device is cylindrical, the aforementioned values may refer to the diameter of the channel. In this respect, an inlet channel and/or an outlet channel may have a channel diameter of from about 0.5 microns to about 1 millimeter, including all increments of about 0.5 microns therebetween.

Generally, an inlet channel and/or an outlet channel of the microfluidic device may have a throat width of from about 0.5 microns to about 1 millimeter, including all increments of about 0.5 microns therebetween. For instance, an inlet channel and/or an outlet channel may have a throat width of about 0.5 microns or more, such as about 1 micron or more, such as about 5 microns or more, such as about 10 microns or more, such as about 15 microns or more, such as about 20 microns or more, such as about 25 microns or more, such as about 30 microns or more, such as about 40 microns or more, such as about 50 microns or more, such as about 100 microns or more, such as about 150 microns or more, such as about 200 microns or more, such as about 300 microns or more, such as about 400 microns or more, such as about 500 microns or more. In general, an inlet channel and/or an outlet channel may have a throat width of about 1 millimeter or less, such as about 500 microns or less, such as about 400 microns or less, such as about 300 microns or less, such as about 200 microns or less, such as about 150 microns or less, such as about 100 microns or less, such as about 50 microns or less, such as about 40 microns or less, such as about 30 microns or less, such as about 25 microns or less, such as about 20 microns or less, such as about 15 microns or less, such as about 10 microns or less, such as about 5 microns or less. As used herein, the “throat” is the portion of an inlet channel and/or an outlet channel having a narrowed or expanded geometry as compared to the rest of the inlet channel and/or an outlet channel. For instance, as illustrated in FIG. 20, the throat 78 of the outlet channel 74 may have a narrowed geometry. It should be understood that the width of the throat refers to the narrowest width of the throat (i.e., the smallest width of the throat) for a throat having a narrowed geometry and refers to the most expanded width of the throat (i.e., the largest width of the throat) for a throat having an expanded geometry respectively. Notably, when the throat of an inlet channel and/or an outlet channel is cylindrical, the aforementioned values may refer to the diameter of the throat. In this respect, an inlet channel and/or an outlet channel may have a throat diameter of from about 0.5 microns to about 1 millimeter, including all increments of about 0.5 microns therebetween. It should be understood that the diameter of the throat refers to the narrowest diameter of the throat (i.e., the smallest diameter of the throat) for a throat having a narrowed geometry and refers to the most expanded diameter of the throat (i.e., the largest diameter of the throat) for a throat having an expanded geometry respectively.

Notably, in some aspects, the throat width of an inlet channel and/or an outlet channel having a throat may be larger than the width of one or more inlet channels and/or one or more outlet channels of a microfluidic device. In general, the ratio of the throat width of an inlet channel and/or an outlet channel to an inlet channel width and/or an outlet channel width may be from about 40:1 to about 1:40, including all incremental ratios therebetween. For instance, the ratio of the throat width of an inlet channel and/or an outlet channel to an inlet channel width and/or an outlet channel width may be about 40:1 or less, such as about 30:1 or less, such as about 20:1 or less, such as about 15:1 or less, such as about 10:1 or less, such as about 5:1 or less, such as about 4:1 or less, such as about 3:1 or less, such as about 2:1 or less, such as about 1:1 or less, such as about 1:2 or less, such as about 1:3 or less, such as about 1:4 or less, such as about 1:5 or less, such as about 1:10 or less, such as about 1:15 or less, such as about 1:20 or less, such as about 1:30 or less. In general, the ratio of the throat width of an inlet channel and/or an outlet channel to an inlet channel width and/or an outlet channel width may be about 1:40 or more, such as about 1:30 or more, such as about 1:20 or more, such as about 1:10 or more, such as about 1:5 or more, such as about 1:4 or more, such as about 1:3 or more, such as about 1:2 or more, such as about 1:1 or more, such as about 2:1 or more, such as about 3:1 or more, such as about 4:1 or more, such as about 5:1 or more, such as about 10:1 or more, such as about 15:1 or more, such as about 20:1 or more, such as about 30:1 or more. Notably, when the throat and one or more inlet channels and/or one or more outlet channels are cylindrical, the aforementioned values may refer to the diameter of the throat and the one or more inlet channels and/or one or more outlet channels. In this respect, the ratio of the throat diameter of an inlet channel and/or an outlet channel to an inlet channel diameter and/or an outlet channel diameter may be from about 40:1 to about 1:40, including all incremental ratios therebetween.

In general, an inlet channel and/or an outlet channel may have a length from about 300 microns to about 10 cm, including all increments of 1 micron therebetween. Generally, an inlet channel and/or an outlet channel may have a length of about 300 microns or more, such as about 500 microns or more, such as about 1000 microns or more, such as about 5000 microns or more, such as about 1 cm or more. In general, an inlet channel and/or an outlet channel may have a length of about 10 cm or less, such as about 5 cm or less, such as about 1 cm or less, such as about 5000 microns or less, such as about 1000 microns or less.

Generally, an inlet channel may have an inlet channel stream flowing or moving therein. In general, an outlet channel may have an outlet channel stream flowing or moving therein. Notably, an inlet channel stream and/or an outlet channel stream may have a flow pattern of co-flows, threading, plugging, dripping, jetting, multi-satellite, or a combination thereof. In general, co-flows refers to the parallel movement of two or more immiscible fluids, from separate streams, that flow into the same channel without significant intermixing. Further, threading refers to the formation of threads of one fluid within another. Additionally, plugging refers to the formation of well-defined droplets of one fluid within another fluid. Notably, the well-defined droplets have a volume that occupies the entire width and/or height of a channel. In this respect, the droplets of a plugging flow pattern “plug” the channel. Further, multi-satellite refers to the formation of a plurality of smaller droplets, which may be referred to as satellites, around a larger, central droplet. In addition, dripping refers to when a fluid exits an opening, such as a nozzle, and continuously forms droplets that do not extend the entire width and/or height of a channel. In this respect, the droplets are surrounded by a fluid. Moreover, jetting refers to when a fluid exits an opening, such as a nozzle, in a continuous stream.

In some aspects, a channel (e.g., an outlet channel) may have a channel stream (e.g., an outlet channel stream) comprising a plurality of liquid droplets. Generally, the configuration and design of the channels, which include channel geometry, channel angle (e.g., inlet channel junction angle), and the configuration and design of the junction may influence the droplet size and/or the rate of droplet generation. In this respect, the configuration and design of the channels and/or the junction may be configured to influence the flow pattern, droplet size, and/or the rate of droplet generation.

Notably, an inlet channel stream (e.g., gas inlet channel stream, liquid inlet channel stream) and/or an outlet channel stream (e.g., gas outlet channel stream, liquid outlet channel stream) may have a volumetric flow rate from about 0.01 μL/min to about 150 mL/min, including all increments of about 0.01 μL/min therebetween. For instance, an inlet channel stream and/or an outlet channel stream may have a volumetric flow rate of about 0.01 μL/min or more, such as about 0.1 μL/min or more, such as about 0.5 μL/min or more such as about 1 μL/min or more, such as about 2 μL/min or more, such as about 3 μL/min or more, such as about 4 μL/min or more, such as about 5 μL/min or more, such as about 6 μL/min or more, such as about 7 μL/min or more, such as about 8 μL/min or more, such as about 9 μL/min or more, such as about 10 μL/min or more, such as about 15 μL/min or more, such as about 1 mL/min or more, such as about 5 mL/min or more, such as about 10 mL/min or more, such as about 15 mL/min or more, such as about 20 mL/min or more, such as about 30 mL/min or more, such as about 40 mL/min or more, such as about 50 mL/min or more, such as about 80 mL/min or more, such as about 100 mL/min or more. In general, an inlet channel stream and/or an outlet channel stream may have a volumetric flow rate of about 150 mL/min or less, such as about 100 mL/min or less, such as about 80 mL/min or less, such as about 50 mL/min or less, such as about 40 mL/min or less, such as about 30 mL/min or less, such as about 20 mL/min or less, such as about 15 mL/min or less, such as about 10 mL/min or less, such as about 5 mL/min or less, such as about 1 mL/min or less, such as about 15 μL/min or less, such as about 10 μL/min or less, such as about 9 μL/min or less, such as about 8 μL/min or less, such as about 7 μL/min or less, such as about 6 μL/min or less, such as about 5 μL/min or less, such as about 4 μL/min or less, such as about 3 μL/min or less, such as about 2 μL/min or less, such as about 1 μL/min or less.

In general, the microfluidic device and/or any component thereof (e.g., inlet channel, outlet channel) may comprise polydimethylsiloxane, a glass (e.g., pyrex, quartz, fused silica, alkali-free glass, soda lime glass), silicon, steel, acrylic, a ceramic material, or a combination thereof. It should be understood that the microfluidic device and/or any component thereof may include a combination of one or more glasses and/or one or more ceramic materials. It should be understood that the microfluidic cartridge and/or any component thereof may include a combination of one or more glasses and/or one or more ceramic materials. Notably, the aforementioned materials may be used for their ability to withstand high stresses. In this respect, the aforementioned materials may have a preferable Young's modulus. In some aspects, when glass (e.g., quartz) is utilized to form at least one component of the microfluidic device, the respective component of the microfluidic device may be treated with a silane (e.g., a long carbon chain) such that the silane anchors or attaches to the surface of the glass and makes the glass surface hydrophobic or hydrophilic. Notably, the glass surface may include at least some hydrophobic alkyl groups clustered together on the surface of the glass, which may develop a surface that may form and/or comprise micelles.

In general, a system formed in accordance with the present disclosure may include a mobile analysis device, one or more microfluidic devices, one or more microfluidic cartridges, one or more containers or reservoirs, one or more spectrophotometric devices, one or more heating sources, one or more cooling sources, one or more microfluidic device chambers, one or more microfluidic cartridge chambers, or a combination thereof. In some aspects, one or more microfluidic cartridges and/or one or more microfluidic devices may be positioned or placed in a mobile analysis device. The area or space in which a microfluidic cartridge or microfluidic device is positioned or placed in a mobile analysis device may be referred to as a microfluidic cartridge chamber or a microfluidic device chamber respectively. In general, the mobile analysis device may comprise one or more microfluidic devices, one or more microfluidic cartridges, one or more containers or reservoirs, one or more spectrophotometric devices, one or more heating sources, one or more cooling sources, one or more microfluidic device chambers, one or more microfluidic cartridge chambers, or a combination thereof. In this respect, the mobile analysis device may be an all-in-one device for the mixing and detecting of chemical, biological and/or radiological/nuclear (CBRN) threats. The one or more microfluidic cartridges and/or microfluidic devices may be replaceable or exchangeable. In this respect, after a microfluidic cartridge and/or microfluidic device has been utilized, the used microfluidic cartridge and/or microfluidic device may be ejected or removed from a microfluidic cartridge chamber or microfluidic device chamber of the mobile analysis device, and another microfluidic cartridge and/or microfluidic device may be positioned or placed in the microfluidic cartridge chamber and/or microfluidic device chamber of the mobile analysis device. In this respect, a mobile analysis device formed in accordance with the present disclosure may be able to analyze any number of samples, such as any number of mixtures of a sensing solution and an analyte solution. The mobile analysis device may be configured to measure and/or analyze one or more streams (e.g., one or more inlet channel streams) and/or one or more stream mixtures (e.g., a mixture of a sensing solution and an analyte solution) in a microfluidic device, a microfluidic cartridge, and/or a container or reservoir. In this respect, the mobile analysis device may comprise one or more spectrophotometric devices (e.g., a spectrofluorometer) configured to analyze one or more streams, one or more stream mixtures, and/or one or more containers or reservoirs. Generally, a temperature gradient may be applied to a microfluidic device in a mobile analysis device. In this respect, in one aspect, a temperature gradient may be applied by a heating source (e.g., a hot plate, a heating coil, etc.) or a combination of heating sources that are incorporated into the mobile analysis device and/or a cooling source (e.g., Peltier cooling device, a cooling tube, etc.) or a combination of cooling sources that are incorporated into the mobile analysis device.

Notably, a microfluidic device or microfluidic cartridge formed in accordance with the present disclosure may be reusable or single-use. In general, a reusable microfluidic device or microfluidic cartridge and/or a single-use microfluidic device or microfluidic cartridge may utilize multiplexed Förster Resonance Energy Transfer based (FRET-based) and/or Nanometal Surface Energy Transfer based (NSET-based) sensors capable of simultaneous detection of multiple threats, including one or more CBRN threats. In general, a reusable microfluidic device or microfluidic cartridge may be cleaned after use. In this respect, one or more channels of a reusable microfluidic device or microfluidic cartridge may be flushed or cleaned with a cleaning solution after the microfluidic device or microfluidic cartridge is used to mix and/or detect one or more CBRN threats. Generally, one or more channels of a single-use microfluidic device or microfluidic cartridge may be flushed or cleaned with a cleaning solution after the microfluidic device or microfluidic cartridge is used to mix and/or detect one or more CBRN threats. Alternatively, one or more channels of a single-use microfluidic device or microfluidic cartridge may not be flushed or cleaned with a cleaning solution after the microfluidic device or microfluidic cartridge is used to mix and/or detect one or more CBRN threats. Generally, a single-use microfluidic device or microfluidic cartridge may be disposed of after its use to mix and/or detect one or more CBRN threats or after it is cleaned, such as via a cleaning solution.

Generally, a system incorporating a microfluidic device and/or microfluidic cartridge formed in accordance with the present disclosure may have two or more microfluidic devices and/or two or more microfluidic cartridges in series and/or in parallel. In some aspects, a system incorporating a microfluidic device or microfluidic cartridge formed in accordance with the present disclosure may utilize clamps for the positioning or placement of a microfluidic device or a microfluidic cartridge.

Notably, one or more nozzles of a system may be placed in fluid communication with one or more inlet channels and/or one or more outlet channels of a microfluidic device. The one or more nozzles may introduce a stream, such as a gas stream, a liquid stream, or a combination thereof, into one or more inlet channels of a microfluidic device. In general, the one or more nozzles may be removed and the one or more inlet channels and/or one or more outlet channels may be sealed or capped. Notably, the sealing or capping of a microfluidic device or microfluidic cartridge may allow for the transportation of a microfluidic device containing one or more CBRN threats. After a microfluidic device or microfluidic cartridge is removed from a system, another microfluidic device or microfluidic cartridge may positioned or placed in the system and replace the previous microfluidic device or microfluidic cartridge. Alternatively, as previously disclosed herein, a microfluidic device or microfluidic cartridge may be reusable and may be cleaned, as opposed to being removed.

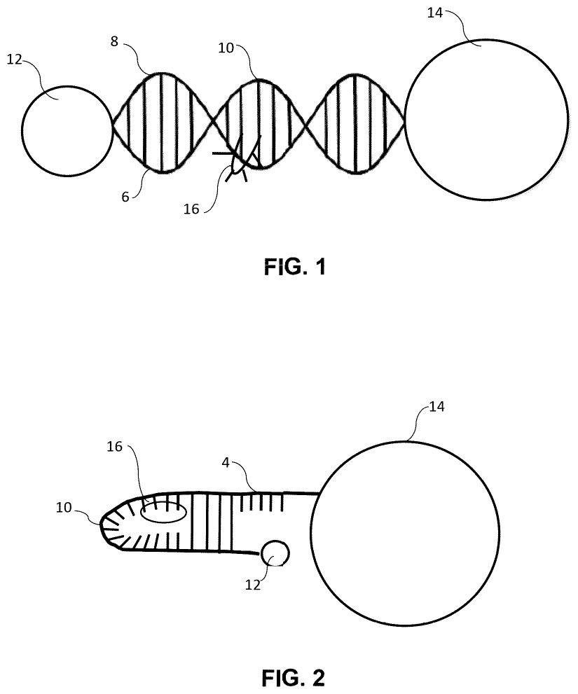

Disclosed sensors are based upon the energy transfer from an excited donor molecule to an acceptor through nonradiative dipole-dipole interactions. As illustrated in FIG. 1 and FIG. 2, disclosed sensors can include a linking agent 10 between a donor 12 and an acceptor 14. The linking agent 10 includes one or more binding ligands 16 that are specific for a particular analyte. The sensors disclosed in U.S. Patent Publication No. 2023/0221306, which is incorporated herein by reference in its entirety, may be utilized in the sensing solution of the present disclosure.

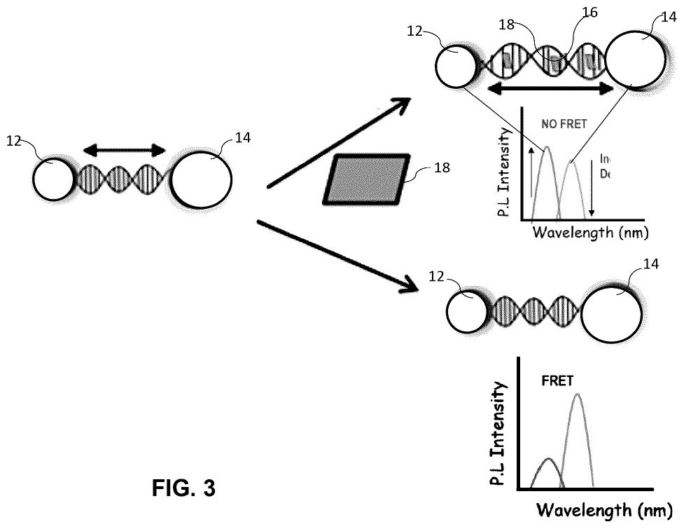

As illustrated in FIG. 3, upon specific binding of an analyte 18 (biological, chemical, radionuclide) with its specific binding ligand 16, a change in conformation of the sensor occurs, e.g., a change in the distance between the acceptor 14 and donor 12, which leads to a change in energy transfer efficiency. This change causes an alteration in the energetic coupling of the two, which causes an optically detectable signal to be emitted or emanate from the sensor. Notably, one or more detectable signals (e.g., first detectable signal, second detectable signal, third detectable signal) may be emitted or emanate from one or more regions (e.g., first region, second region, third region) of a sensor. An optically detectable signal can be, for example, a quenching of an emission, an increase in intensity of an emission, a change in wavelength of an emission, or any combination thereof, depending upon the specific characteristics of the donor, the acceptor, and the change in relationship between the two upon the analyte binding. For instance, in the example illustrated in FIG. 3, upon binding of the analyte 18 to the specific binding ligand 16, the distance between the donor 12 and the acceptor 14 is increased. This, in turn, leads to an increase in the photoluminescent (PL) intensity of the donor emission and a decrease in the PL intensity of the acceptor as compared to no analyte binding due to a decrease in FRET response from the increased distance.

The detectable response of a particular sensing pair 12, 14 upon binding of a CBRN threat 18 to a binding ligand 16 of a linking agent 10 can be utilized to identify a particular type of threat. For example, different types of threats can be identified by a different optically detectable response (e.g., increased, quenched, or otherwise altered emission) upon analyte binding to a particular sensing pair of a sensor, leading to a unique indicator of the FRET-based biosensor. Moreover, in the case of binding multiple threats to a single sensor, a different response can be obtained as compared to the responses for each threat individually.

In implementation, a sensor can utilize FRET-based sensing, NSET-based sensing, or a combination thereof. The primary difference between FRET-based sensing and NSET-based sensing is the distance range of the sensing pairs. FRET-based sensing pairs are generally designed for a distance range between the donor and acceptor of from about 1 to about 20 nm, while NSET can extend that range to about 40 nm. In addition, for FRET, both the donor and acceptor are considered as point dipoles in which an excited donor (e.g., a molecule, molecular complex, ion, or nanoparticle) transfers the excitation energy non-radiatively to an acceptor, and the process is driven by dipole-dipole interactions between the transition dipole moment of donor emission and acceptor absorption in energetic resonance. As such, it is necessary to have a spectral overlap between the donor emission and the acceptor absorption. In NSET systems, the acceptor is considered a nanometric surface modeled as a collection of multiple point dipoles. The FRET point dipole-point dipole interaction results in the rate of energy transfer between the two components being dependent on the inverse of the distance to the sixth power, whereas the NSET point dipole-surface dipole interaction results in the rate of energy transfer being dependent on the inverse of the distance to the fourth power. Either system or a combination of both systems can be utilized in disclosed sensors to provide for real-time detection of multiple chemical threats.

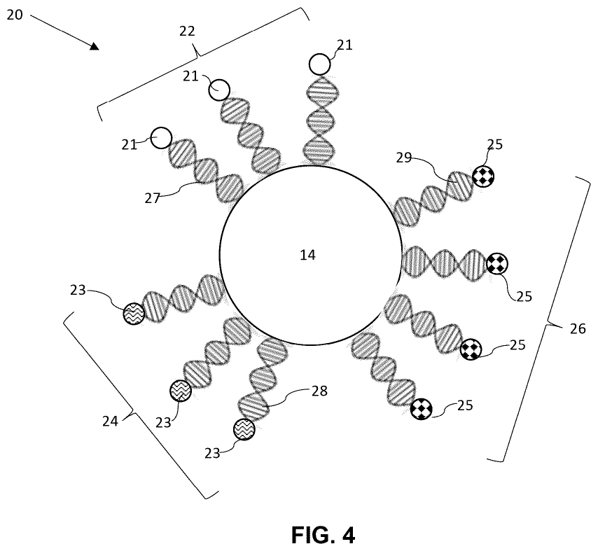

FIG. 4 schematically illustrates a sensor including multiple different sensing pairs. As illustrated in FIG. 4, a multiplexed sensor 20 can include multiple instances of each of several different coupled donor/acceptor pairs 22, 24, 26. In some embodiments, the multiple instances of each of the different donor acceptor pairs 22, 24, 26, can be located in proximity to one another in a predetermined region (e.g., a first region, a second region, a third region) of a sensor 20, as illustrated in FIG. 4. This approach may strengthen the signal strength from any one type of pair 22, 24, 26. However, this is not a requirement of the sensors, and in other embodiments, a sensor can include multiple instances of a coupled pair distributed across an entire sensor and randomly mixed with multiple instances of a different coupled pair.

A sensor can include a single instance of any one of the multiple coupled pairs or multiple instances. For instance, a sensor can include from 1 to about 500 instances of a single type of a coupled pair of a sensor (e.g., from 1 to about 500 instances of coupled pair 22, coupled pair 24, and coupled pair 26 of the sensor of FIG. 4). Moreover, the number of coupled pairs of any one type can be the same or different as compared to the number of other types on a single sensor.

Each of the different coupled donor/acceptor coupled pairs 22, 24, 26 can share either a donor or an acceptor. For instance, in the illustrated embodiment, each of the coupled donor/acceptor pairs 22, 24, 26 can include the same acceptor 14, but a different donor 21, 23, 25, respectively. The component that differs in each of the different coupled pairs can be selected to ensure that the detectable signals of each coupled pair 22, 24, 26 will likewise differ. As such, a detectable signal of a coupled pair 22 that includes a donor 21 and an acceptor 14 will be detectably different as compared to a detectable signal between a coupled pair 24 that includes a donor 23 and the acceptor 14, and so on for each type of coupled pair of a sensor.

Donor/acceptor pairs as may be incorporated in a sensor are not particularly limited, provided each different donor acceptor pair of a single sensor can be identifiably detected from the others upon analyte binding. For instance, any optically active material that can emit light in the range from about 100 nm to about 100 microns (e.g., from ultraviolet to infrared) can be utilized as a donor, such as from about 380 nm to about 25 microns (e.g., from visible light to mid-infrared light), or from about 380 nm to about 10 microns (e.g., from visible light to near-infrared) can be used as a donor in disclosed sensors. In one embodiment, a donor can emit light in the visible spectrum, which may provide for ease of use in an in-field sensing application.

An acceptor for use with any particular donor should be selected so as to provide a detectable change in the emission upon the expected variation in conformation between the two upon analyte binding. One criterion that can be considered in determining suitable pairings is the relative emission/fluorescence spectrum of the donor compared to that of the acceptor. The emission spectrum of a donor and the absorbance spectrum of the acceptor should be in suitable range to one another (e.g., overlapping) such that the emission spectrum of the donor is at a wavelength that is able to interact with the absorbance spectrum of the acceptor at a first proximity and orientation relative to the acceptor and that upon change in that proximity and orientation due to analyte binding, the emission of the coupled pair is altered and thereby provide for an optically detectable signal signifying the analyte binding.

By way of example, when the donor is excited, for instance by visible light, a portion of the energy emitted by the donor can be transferred to the acceptor when the acceptor is spatially close enough to the donor (i.e., the distance between them is approximate to or less than the Forster radius). Once the acceptor absorbs the energy, it in turn fluoresces in its characteristic emission wavelength, resulting in a recognizable emission of the pair. Upon analyte binding, the distance between the two can increase causing a change in the emission of the pair (e.g., quenching of the acceptor fluorescence or acceptor signal) and an optically detectable change in the emission.

In some embodiments, upon binding a target analyte, the detectable signal can be the quenching of a signal as compared to no analyte binding. For instance, a signal of a donor/acceptor pair can be quenched by at least 1%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100%, including all percentages in between each percentage listed.

In some embodiments, a sensor can include a nanoparticle of a plasmonic metal as a donor or an acceptor of a system, e.g., a plasmonic metal particle, such as a plasmonic metal nanoparticle, of gold, silver, copper, platinum, nickel, or palladium as an acceptor or a donor of a sensor. A metallic component is not limited to these materials, however, and other metals as may be incorporated in a system can include but are not limited to lithium, sodium, copper, aluminum, magnesium, barium, potassium, rubidium, and cesium. A particle may comprise mixtures or combinations of metal atoms. Generally, the metallic component may be a noble metal. A nanoparticle component can generally have a largest cross-sectional dimension (e.g., a diameter) of about 1 nm or greater, such as from about 1 nm to about 100 nm, such as from about 1 nm to about 80 nm, such as from about 1 nm to about 50 nm, such as from about 5 nm to about 100 nm, such as from about 5 nm to about 50 nm, such as from about 10 nm to about 50 nm, such as from about 10 nm to about 30 nm.

In some embodiments, a metal nanoparticle component can be the donor or acceptor component that is shared among the multiple coupled donor/acceptor pairs of a sensor. For instance, a gold nanoparticle can be a common acceptor for all of the coupled pairs of a system.

Another class of energy transfer components as may be incorporated in a sensor can include semiconductors formed on a nanometer scale, generally referred to as quantum dots. Exemplary quantum dot materials can include, without limitation, binary materials such as CdTe, CdSe, CdS, ZnSe, PbS, PbSe, ternary materials such as ZnCdSe, CdSeS, CdPbTe, quaternary materials such as ZnCdSSe, as well as doped materials such as Mn doped ZnSe or Mn doped ZnS. In embodiments, a quantum dot of a system can include a core/shell quantum dot, e.g., a quantum dot including a CdSe core and a ZnS shell.

Quantum dots may be employed in a system as donors, acceptors, or both. For instance, a quantum dot of a first type may be utilized as a common donor or acceptor for a system and this common component can be coupled to a plurality of different quantum dots or other types of materials (or a combination thereof) to form a sensor.

The emission of quantum dot components incorporated in a system as a donor can be controlled through size of the particle or, in the case of a core/shell quantum dot, size of the core and/or thickness (i.e., number of atomic layers) of the shell.

Energy transfer components can also include organic molecular components such as fluorescent dyes, proteins, or peptides. Non-limiting examples of a fluorescent organic dyes include fluorescein and its derivatives, rhodamine and its derivatives, Texas Red, Cy5, acridine orange, 2,7-dichlorofluorescein, eosin, rose bengal, 1,2-dihydroxyanthraquinone, 1,4-dihydroxyanthraquinone, 1,8-dihydroxyanthraquinone, 1,3,8-trihydroxy-6-ethylanthraquinone, 1,2,5,8-tetrahydroxyanthraquinone, 1-aminonaphthalene, and 2-aminonaphthalene. Fluorescent derivatives of any of the foregoing are further examples of suitable fluorophores. The foregoing are merely examples and are not intended to be exhaustive. Notably, organic molecular components may be referred to as organic molecules.

A fluorescent protein may be, for example, a green fluorescent protein (e.g., TurboGFP, Azami Green, ZsGreen, Emerald), a blue fluorescent protein (e.g., EBFP2, mTagBFP2), a yellow fluorescent protein (mYFP, mVenus, mCitrine, TurboYFP), a red fluorescent protein (TurboRFP), mRuby2, mStrawberry, FusionRed). Fluorescent peptides are also embraced herein.

Each of the donor and acceptor can include suitable derivatization so as to be covalently or otherwise bonded to a coupling agent. For instance, a nanoparticle (a metal nanoparticle or a quantum dot) may include a coating on a surface that includes a polymer that can provide a hydrophobic, hydrophilic, or otherwise charged surface, or providing functional groups or groups that can subsequently be modified to provide functional groups that can directly or indirectly bind the desired coupling agent. A molecular donor or acceptor can be derivatized to include a functional group or a group that can subsequently be modified to include a functional group that can directly or indirectly bind the desired coupling agent. For instance, a donor and acceptor of a system can be functionalized to include a carboxyl, amine, or sulfhydryl group that can covalently bind an amine, carboxyl, or disulfide of a polynucleotide coupling agent under suitable reactive conditions, e.g., by reduction of a disulfide and subsequent binding to a sulfhydryl of an energy transfer component. Other suitable ligand exchange and coupling chemistries are known in the art as may be utilized.

Referring again to FIG. 1 and FIG. 2, each donor/acceptor pair 12, 14 can be physically retained to one another by use of a linking agent 10 that includes a polynucleic acid. As utilized herein, the term “polynucleic acid” is synonymous with the term “polynucleotide” and refers to a polymer that includes natural bases and backbones as well as a polymer including one or more modified bases and/or backbones. Thus, a polynucleic acid linking agent as encompassed herein can include a backbone modified for stability or for any other purpose. A polynucleotide of a linking agent can include pyrimidine or purine bases such as those bases characteristic of guanine, adenine, thymine, and cytosine. However, other purine or pyrimidine bases may be employed in a polynucleic acid of a linking agent, such as inosine, or modified bases, such as tritylated bases, to name just two examples. It will be appreciated that a great variety of modifications have been made to natural components of polynucleic acids that serve many useful purposes known to those of skill in the art. The term “polynucleotide(s)” as it is employed herein embraces such chemically, enzymatically, or metabolically modified forms of polynucleotides, as well as the synthetic forms of polynucleotides. The term also embraces short polynucleotides often referred to as oligonucleotide(s).

A linking agent can include a double stranded polynucleic acid as in FIG. 1 or a single-stranded polynucleic acid as in FIG. 2. For instance, in an embodiment as illustrated in FIG. 1, a first polynucleotide 8 can be coupled to the donor 12 and a second polynucleotide 6 can be coupled to the acceptor 14. The first and second polynucleotides 6, 8 can be mated along at least a portion of their respective lengths so as to hybridize to one another and couple the donor 12 to the acceptor with a double stranded linking agent 10.

The length of a polynucleotide linking agent can allow energy transfer to occur at least in the non-analyte bound conformation. For instance, a polynucleotide linking agent 10 having a length of about 100 bases or less, e.g., from about 5 to about 100 bases, from about 10 to about 90 bases, or from about 15 to about 85 bases, such as 5, 10, 15, 20, 25, 30, 40, 60, 80, or any number of bases therebetween in length can allow for energy transfer to occur and provide stability to a sensor.

When considering a single stranded linking agent 10 as in FIG. 2, a first end of a polynucleotide 4 can be coupled to a donor 12 and a second end of the polynucleotide 4 can be coupled to the acceptor 14. As indicated, in one embodiment, a first portion of the single stranded linking agent 10 can be complementary and configured to hybridize with a second portion of the single stranded linking agent 10, upon which the donor and acceptor can be brought into suitable proximity for optically detectable energy transfer.

As used herein, the terms “complementary” is used in reference to polynucleotides related by the base-pairing rules. For example, the sequence “5′-A-G-T-3′,” is complementary to the sequence “3′-T-C-A-5′.” Complementarity may be “partial,” in which only some bases of a polymer are matched according to the base pairing rules.

As used herein, the term “hybridization” is used in reference to the pairing of complementary nucleic acids. Hybridization and the strength of hybridization (i.e., the strength of the association between the nucleic acids) is influenced by such factors as the degree of complementarity between the nucleic acids, stringency of the conditions involved, and the thermodynamics of the formed hybrid. “Hybridization” methods involve the non-covalent bonding of one nucleic acid to another, complementary nucleic acid.

The linking agent 10 of each acceptor/donor pair of a system can also include one or more binding ligands 16. The binding ligand of each pair is specific for a particular analyte that represents a CBRN threat. For instance, an analyte can be the threat itself, e.g., a radiological threat such as strontium, cesium, etc. as well as a metabolite or adduct of a threat, e.g., a sarin metabolite, an anthrax metabolite, etc. In some embodiments, two or more binding ligands for each analyte of interest can be added to a sensor for secondary positive identification. Such an approach can decrease the risk of false positives for more reliable detection.

In various embodiments, the selective binding of the binding ligand 16 to a particular CBRN threat can be an innate component of the linking agent 10. For instance, a polynucleotide linking agent 10 can include a binding ligand 16 as an innate portion of the polynucleotide structure, e.g., a portion of the linking agent sequence, e.g., less than about 10 bases of the complete linking agent 10, that is a specific binding agent for the analyte. For example, as illustrated in FIG. 1 a length of several bases (e.g., from about 3 to about 6 bases in length) of one of the strands of the double stranded linking agent 10 can form a binding ligand 16 that extends from one or the other strands of the double stranded linking agent 10, e.g., a non-hybridized portion of the linking agent 10. In the case of a single stranded linking agent 10, as in FIG. 2, a portion of the linking agent 10 can likewise provide the binding ligand 16. In either case, in the presence of the targeted analyte the binding ligand 16 will preferentially bind the analyte and change the distance (either increase or decrease) between the donor 12 and the acceptor 14 and thereby modify the energy transfer between the two.

In some embodiments, the selective binding of a binding ligand 16 to a particular CBRN threat can be provided through addition of a binding functionality to a pre-formed linking agent 10, for instance through modification of a single base or a series of bases of a linking agent to include one or more inserted sequences that provides a specific binding ligand 16.

In some embodiments, in the absence of an analyte, a binding ligand 16 can hybridize with a complementary segment of a linking agent 10. In general, however, in the absence of the analyte a binding ligand 16 can be present in the linking agent 10 as a non-hybridized portion, as illustrated in FIG. 1 and FIG. 2.

In one embodiment, a binding ligand 16 can be a component of an aptamer designed or identified to exhibit specific binding with an analyte of interest. A large number of aptamers have been developed and are known to those of skill in the art and as such are not enumerated herein. For example, aptamers as have been developed to exhibit specific binding to sarin and other nerve agents, anthrax (e.g., PA63, anthrax protective antigen, etc.), organophosphates (e.g., 2 (diisopropylamino) ethyl] methylphosphonothiolate), botulism, metals (e.g., lead, uranium, etc.), any of which could be incorporated in a sensor as disclosed herein.

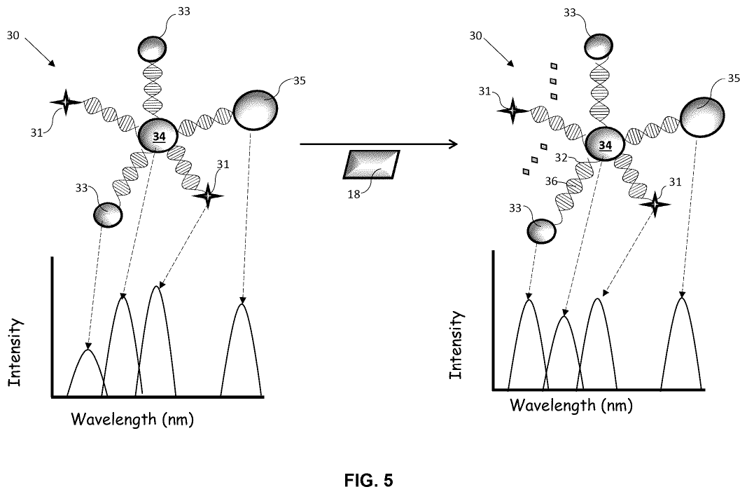

FIG. 5 schematically illustrates the reaction of a multi-functional sensor 30. As illustrated, the sensor 30 includes a common acceptor 34 linked to a plurality of different donors 31, 33, 35. Upon binding of an analyte 18 to a specific binding ligand 36 of one of the linking groups, e.g., the linking group 32 between donor 33 and acceptor 34, the energetic coupling of the pair 33, 34 is altered, leading to an increase in the emission intensity of the donor 33 and the quenching of emission from the acceptor 34. Due to overlap in spectral characteristics of the acceptor 34 with the donor 31, this can also lead to a decrease in emission intensity of the donor 31. Analysis of the spectral response of the sensor 30 can inform a user as to the particular threat encountered by the user or, in other embodiments, can provide more generalized information and inform a user merely that at least one of the analytes tested for by the sensor is present. Through inclusion of multiple different sensing pairs on a single sensor, as schematically illustrated in FIG. 5, the disclosed sensors can allow for simultaneous detection of multiple different biological, chemical, and radiological/nuclear threats. As such, the sensor configuration can include one or more detectors for biological threats, one or more detectors for chemical threats, and one or more detectors for radiological/nuclear threats, thereby allowing for simultaneous detection of different threat types.

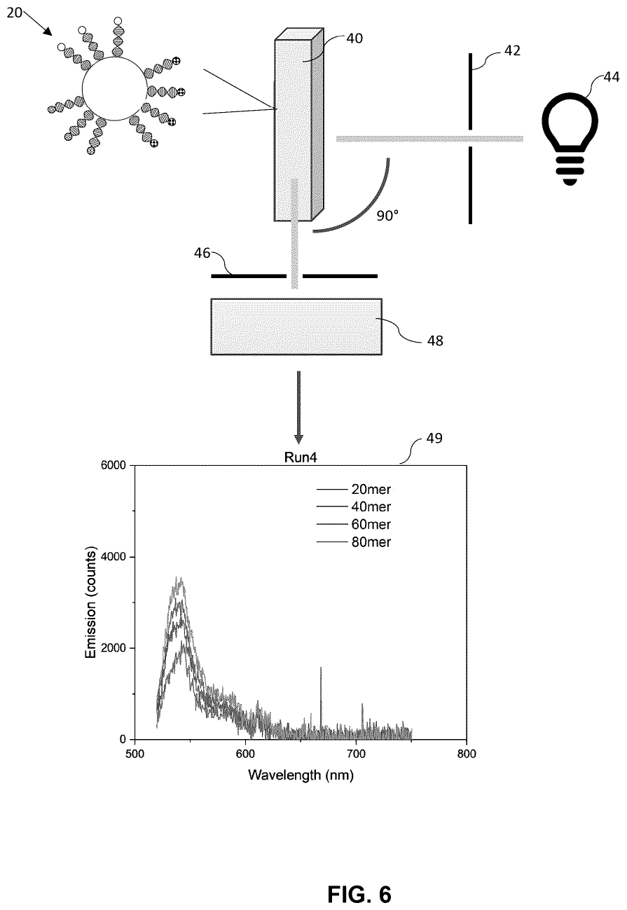

The configuration of a sensing system is not particularly limited. One embodiment of a liquid-based sensing system is illustrated in FIG. 6. In this embodiment, a solution 40 can carry a plurality of individual sensors, e.g., a plurality of a multiplexed sensor 20 as described above. In one embodiment, a sensing solution can be brought into contact with a potential threat agent according to a passive or active design, e.g., a gas that may be suspected of carrying a threat agent (e.g., breathable air of an area) can be passed through the sensing solution. In another embodiment, the sensors 20 can be added to a solution that is suspected of containing one or more of the threat agents of interest. In any case, following combination of the sensors 20 with the threat agents in the solution, the sensing solution 40 can be held in optical communication with a light source 44, e.g., via an excitation monochromator 42 and also in optical communication with a detector 48, e.g., via an emission monochromator 46 at a suitable angle to one another, e.g., 90°. The detector 48 can provide an output 49, e.g., an emission spectrum, that can provide indication of the presence of one or more of the chemical threats capable of detection by the multiplexed sensor 20.

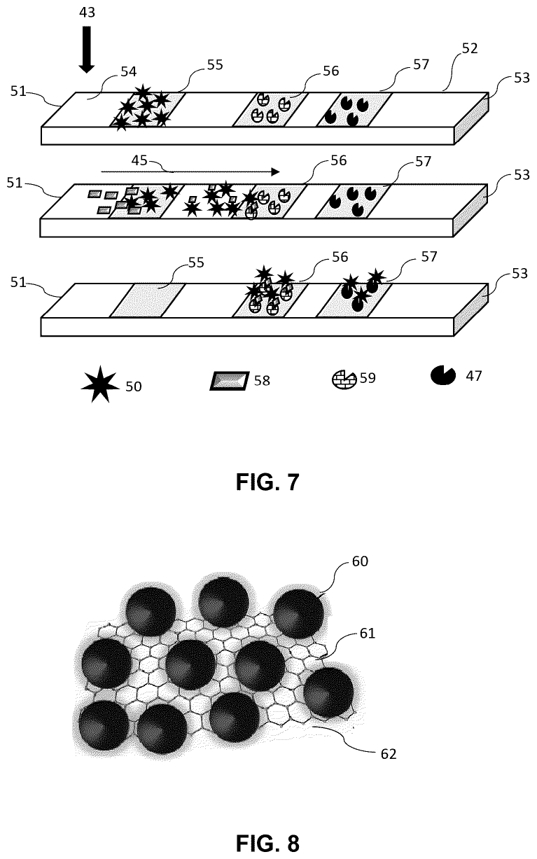

FIG. 7 schematically illustrates another possible embodiment of a sensing system as may incorporate a sensor as disclosed herein. As indicated, in this embodiment, a sensing system can include a lateral flow type configuration. A lateral flow system can include a substrate 52, e.g., a substrate including a porous membrane such as a cellulose-based membrane or the like, which can encourage flow of a liquid sample in a flow direction as indicated from an application end 51 to an indication end 53 of a device.

A lateral flow system can include one or more zones through which a sample can flow, e.g., an application zone 54, a conjugate zone 55, a detection zone 56 and a calibration zone 57. Optionally, a system can include multiple areas within each zone, e.g., multiple conjugation areas that together form a conjugate zone 55, multiple calibration areas of a calibration zone 57, etc. Moreover, in some embodiments, a system need not include all of the zones as are illustrated in the embodiment of FIG. 7. For instance, in some embodiments, a sensing system may not include a calibration zone 57.

To initiate the detection of one or more threat agents 50 within the test sample, a user may directly apply the test sample 43 to a portion of the substrate 52, e.g., to an application zone 54, through which it can then travel to reach one or more detection zones 56 and calibration zones 57. Alternatively, the test sample may first be applied to a sampling pad that is in fluid communication with the substrate 52.

In the illustrated embodiment, the test sample 43 travels from the application zone 54 to a conjugate zone 55 (as shown by the directional arrow 45 in FIG. 7 and the sequential images of the system). The conjugate zone 55 allows the test sample to pass therethrough. The conjugate zone 55 can also contain a plurality of sensors 50 releasably therein. While contained on the conjugate zone 55, these sensors 50 remain available for binding with threat agents 58 contained in the test sample as the test sample passes through the conjugate zone 55. Upon binding with the threat agents 58, the sensors 50 can later serve to identify the presence and type of the analyte(s) 58 in the detection zone 56.

The detection zone 56 may contain an immobilized capture reagent 59. Although not required, it may be desired that the capture reagent 59 be formed from the same class or category of materials (e.g., aptamers) as the specific binding ligands 16 used to form the sensors 50. These capture reagents 59 serve as stationary binding sites for the sensor 50/threat agent 58 complexes. In some instances, the threat agents 58 can be capable of binding multiple specific binding agents (either the same or different from one another). Upon reaching the detection zone 56, one of these binding sites is occupied by the specific binding ligand of the sensor 50/threat agent 58 complex. However, the free binding site of the threat agent 58 can bind to the immobilized capture reagent 59. Upon being bound to the immobilized capture reagent 59, the newly formed ternary complex that includes the sensor 50, the threat agent 58, and the capture reagent 59 signals the presence of the threat agent 58.

As illustrated, a lateral flow-type sensing system can include a calibration zone 57. In this embodiment, the calibration zone 57 is formed on the porous membrane of the substrate 52 and is positioned downstream from the detection zone 56. The calibration zone 57 is provided with a binding agent 47 that is capable of binding to any remaining sensors 50 that pass through the length of the membrane substrate 52. In particular, upon being contacted with the test sample 43, any sensors 50 that do not bind any threat agent 58 migrate through the detection zone 56. In the detection zone 56, as set forth above, the conjugated sensors 50 bind to capture reagents 59 and remain immobilized. However, the unbound sensors 50 and in some embodiments also a portion of the sensor 50/threat agent 58 conjugates continue to migrate through the detection zone 56 and enter the calibration zone 57 of the substrate 52. At the calibration zone 57 these unbound sensors and/or conjugates thereof then bind to the binders 47. When immobilized in the calibration zone 57, any sensors can be detected, and a user can compare the signal intensity in the detection zone 56 to the signal intensity in the calibration zone 57.

The calibration zone 57 may generally provide any number of distinct calibration regions so that a user can better determine the concentration of a particular threat agent 58 within a test sample. In most embodiments, for example, the calibration zone 57 includes two or more calibration distinct calibration regions (e.g., lines, dots, etc.). A comparison may be made between the intensity levels of the calibration regions of a calibration zone 57 and that of the detection zone 56 to calculate the amount and type of threat agents present in the test sample.

A sensing system can carry the individual sensor components according to any known approach. For instance, and as illustrated in FIG. 8, individual sensors can be carried on the surface of a larger carrier, e.g., a particulate or the like. FIG. 8 illustrates a plurality of sensors 60 embedded in a graphene oxide coating 61 over a substrate 62 forming a solid-phase composite that can be brought into contact with a sample suspected of containing one or more threat agents detected by the sensors 60. Optical examination of the surface following contact with a sample can provide information regarding the presence of the threat agents(s) within the sample.

Advanced and portable sensor capabilities for the simultaneous onsite detection of specific CBRN threats as possible with disclosed sensors can significantly limit the risk of exposure to personnel and allow for the rapid, in field collection of essential scientific data and critical evidence. Disclosed sensors can provide effective identification of multiple threats with a much faster, real-time response and a simpler signal readout as compared to previously known CBRN sensors. In this regard, disclosed sensors can be utilized for in field detection due to the low detection limits, small size, and low power requirements.

The present invention may be better understood by reference to the examples set out below.

### EXAMPLES

### Prophetic Example 1

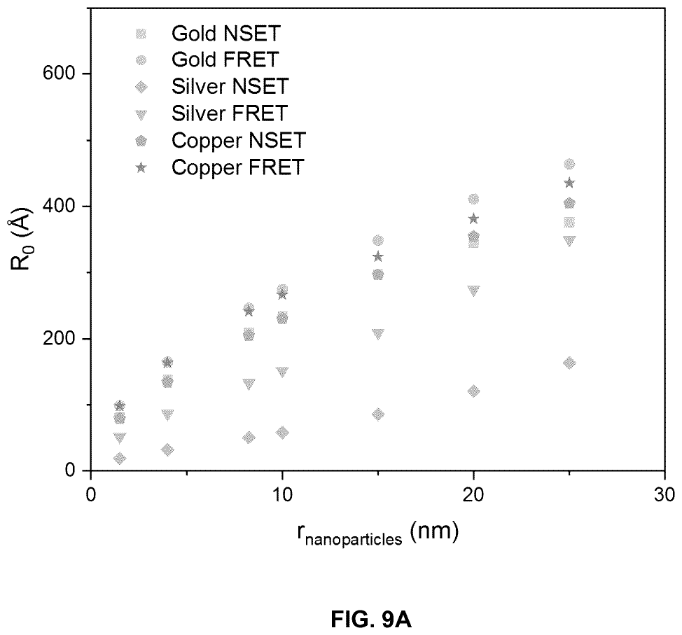

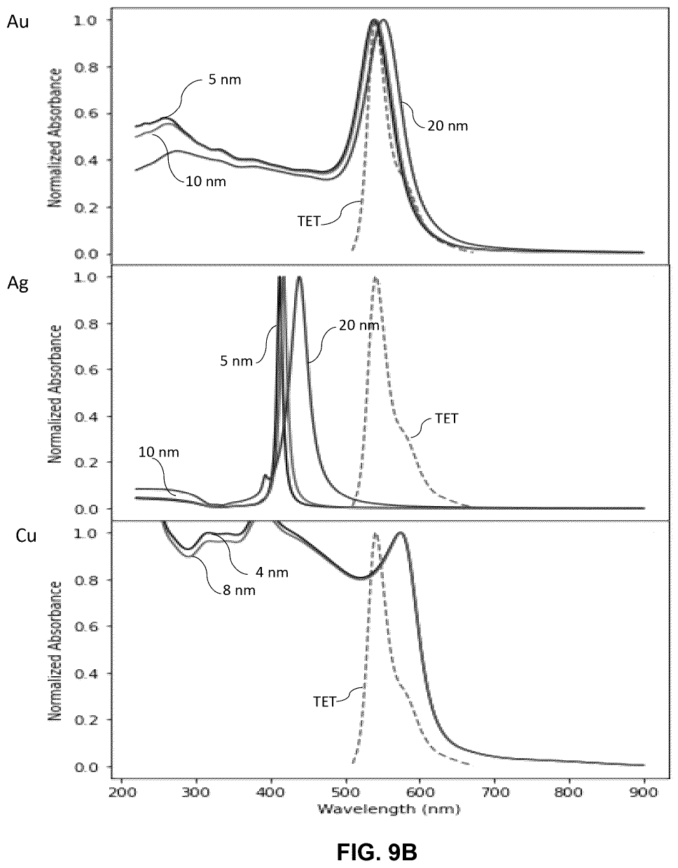

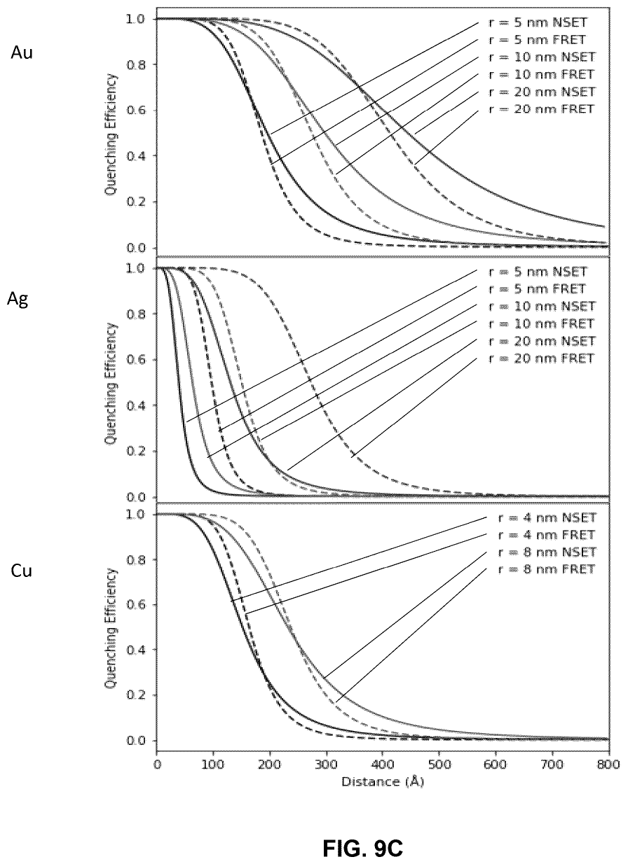

The degree of energy transfer between an energy transfer pair depends upon the distance between the donor and acceptor, as well as on the size and composition of the energy acceptor, which may influence detection limits for detection schemes. To investigate the differences in energy transfer with different metal acceptors, a Python script was created to calculate the theoretical FRET/NSET efficiencies for metal nanoparticles of different sizes and optical properties. FRET/NSET calculations were carried out for 3 different metal nanoparticles (Cu, Ag, Au) between 2-50 nm in diameter. FIG. 9A presents the calculated Forster radii of the different particles. Extinction spectrum for the metal nanoparticle were calculated for both FRET and NSET calculation. Results are provided in FIG. 9B. The emission spectra of an exemplary dye molecule, tetrachlorofluorescein (TET) is overlayed on the extinction spectra of the metal nanoparticles in the figure. The FRET/NSET efficiencies between dyes and given metal nanoparticles for interparticle distances of 4-800 Å were also calculated. Results are shown in FIG. 9C. As indicated, expanding the types of metals used in disclosed sensors can allow for a better control over detection capabilities for biological and chemical sensors.

### Example 1

Gold nanoparticles (AuNPs) were functionalized through ligand exchange with thiol functionalized aptamers.

A first DNA sequence examined was a hairpin sequence such as may be utilized in forming a sensor as schematically illustrated in FIG. 2. This sequence was as follows:

(SEQ ID NO: 1)
5′-ATCCGTCACA CCTGCTCTCG ATGAGACAAG AGGAACACG
GCACAATTGATT TAA-TGGTGTTGGCTCCCGTAT-3

A second DNA sequence examined was a relatively short strand including a single TTTAGT binding site for the sarin metabolite methylphosphonic acid (MePA). The full sequence was as follows:

(SEQ ID NO: 3)
ATTTAGTAGC GGGATCTGTC A

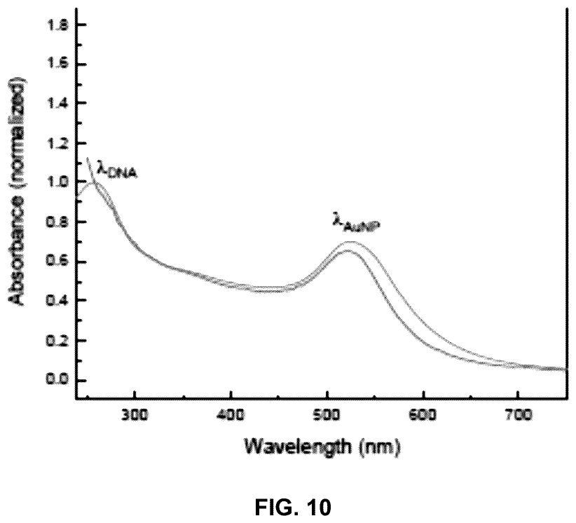

As shown in FIG. 10, the AuNP/DNA conjugates had an additional absorption peak at 260 nm for the DNA, demonstrating successful functionalization.

To verify DNA analyte binding with the second conjugate, the melting temperature of AuNP-ssDNA (SEQ ID NO: 3) was measured in the presence and absence of MePA. The melting temperature (Tₘ) decreased from Tₘ=45° C. to Tₘ=33° C. in the presence of MePA. This indicates interaction and probable binding for the analyte with the DNA strand. This interaction would thus change the distance between the donor and acceptor in a ds-DNA sensor, leading to a detectible change in FRET response.

The AuNP-DNA conjugates that were created with the MePA-targeting aptamers were calculated to have an average of about 70 DNA per AuNP. This high density of targeting ligands was a result of the large nanoparticle surface area, which can lead to more sensitive detection.

### Example 2

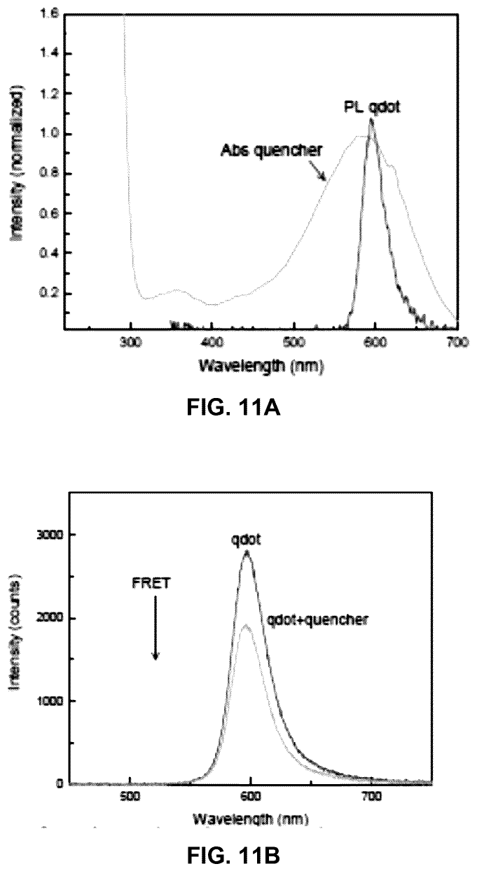

Black Hole Quencher®-2 dye was linked to the surface of a CdSe/ZnS quantum dots (qdot) through dsDNA hybridization of complementary ssDNA conjugated to each component to study the FRET between the qdot donor and the dye quencher acceptor. As shown in FIG. 11A, there was a high degree of spectral overlap between the donor's emission (PL qdot) and the acceptor's absorbance (Abs quencher). After the addition of the complementary strand to the qdot-ssDNA conjugate, the photoluminescence of the qdot decreased as a result of FRET, as depicted in FIG. 11B.

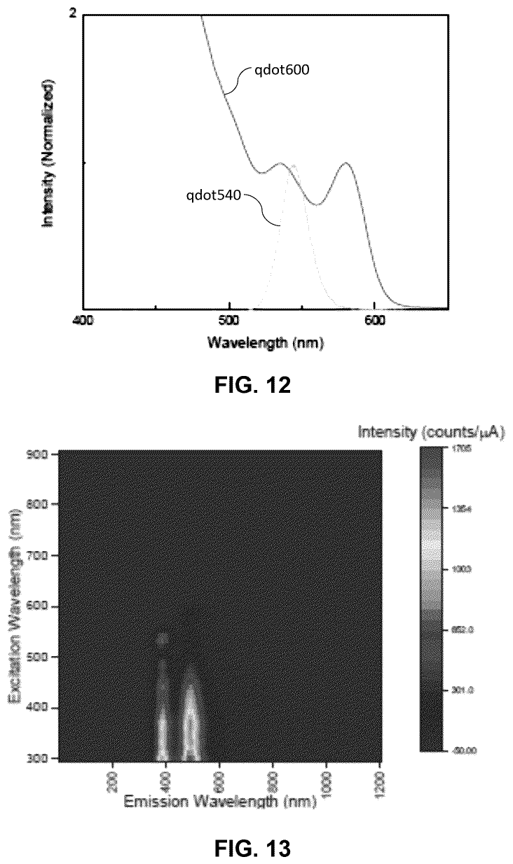

Qdots have the ability to be both FRET donors and acceptors because of their broad absorption spectra and narrow emission peaks. As shown in FIG. 12, there is a high degree of spectral overlap between a qdot with emission at 540 nm (qdot540) and the absorbance of a qdot with emission at 600 nm (qdot600).

One challenge to qdot-qdot FRET pairs is spectral cross talk, which results from the broad qdot absorption profiles. Three-dimensional fluorescence excitation emission matrix (EEM) spectroscopy was measured for a qdot pair without spectral overlap and thus an absence of FRET. The results are shown in FIG. 13. For this FRET pair, there was good spectral separation at all the measured wavelengths. At the higher wavelengths, the acceptor was selectively excited as expected based on the absorbance spectra. EEM spectroscopy allows the measurement of the FRET processes on non-FRET channels, which is above 560 nm for the FRET pair in FIG. 12. When the FRET pairs become more complex, such as in three color FRET conjugates, EEM is a powerful technique for quantifying FRET processes.

### Example 3

CdSe/ZnS quantum dots (qdots) were functionalized with a DNA aptamer specific for the sarin metabolite, methylphosphonic acid (MePA). The aptamer sequence included multiple copies of the TTTAGT binding sequence specific for the MePA. The aptamer sequence was as follows:

(SEQ ID NO: 4)
AACCGGCGTT TAGTGCCTTT AGTCGGTTTA GTGCCTACCG.

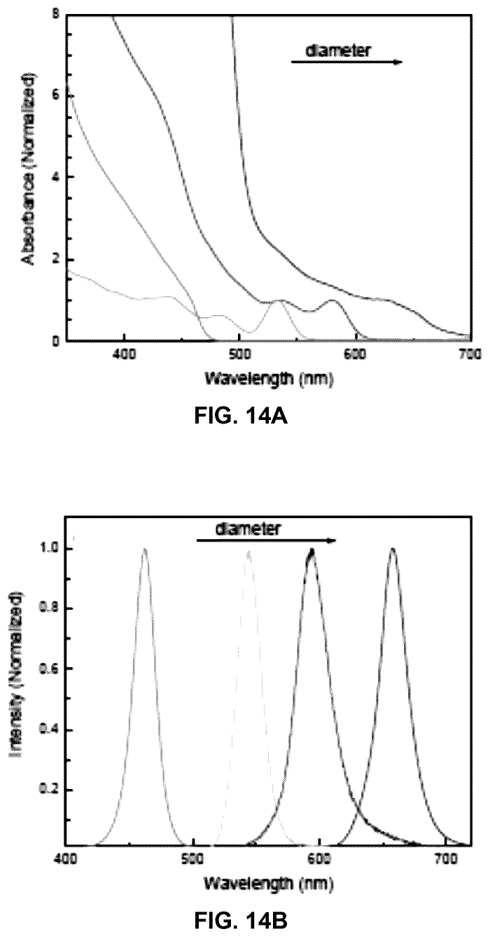

Qdots with different sizes, absorbance and emission wavelengths as illustrated in FIG. 14A (normalized absorbance) and FIG. 14B (normalized intensity) underwent ligand exchange and were dispersed in aqueous buffers where they were then coupled to the aptamer through standard coupling chemistry. After phase transferring, the quantum yield (QY) of the qdots were relatively stable, with QY up to 25% after phase transferring.

FIG. 15 illustrates the results of an investigation of comparison of response of a FRET pair including a CdSe/ZnS qdot donor (D) with a Black Hole Quencher®-2 quenching acceptor (A) with and without SEQ ID NO: 4 DNA conjugation at high concentration. As shown, FRET-based detection was successfully measured through restoration of I/I₀ for the bound D-A pair in the presence of MePA.

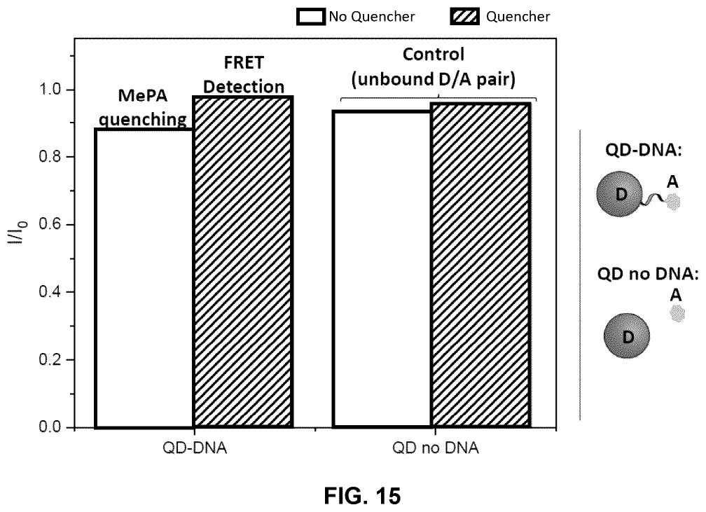

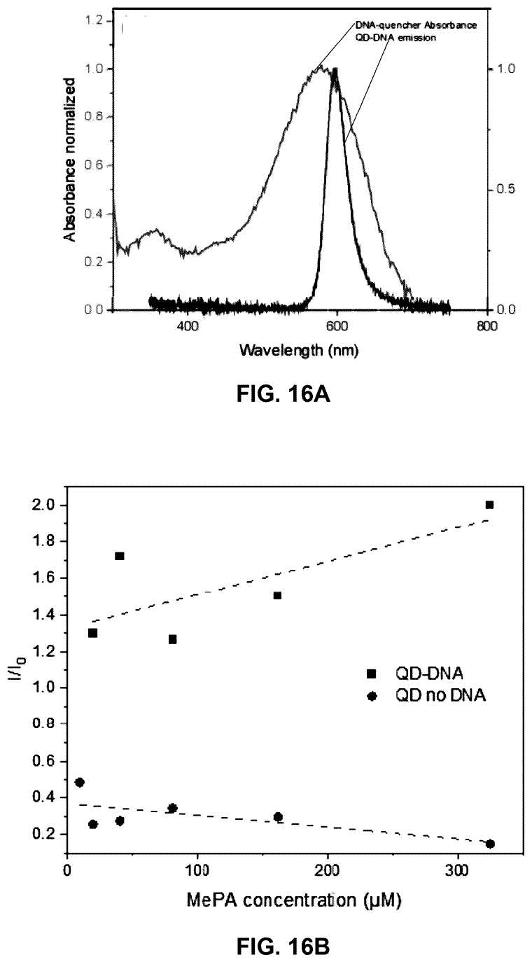

FIG. 16A illustrates the overlap in normalized absorbance for the FRET pair components (a portion of FIG. 11A) and FIG. 16B compares the sensitivity to the presence of MePA for the qdot-quencher FRET pair with the target DNA connection between the FRET components or without. As shown, the sensitivity was much greater for the DNA-separated pair as measured by the ratio of fluorescence measurement (I) for various MePA concentration to fluorescence of the solution with no MePA added (I₀).

### Prophetic Example 2

An anthrax detection sensor as described by FIG. 2 including a fluorescence dye (ATTO488) donor and AuNP FRET pair was envisioned. Anthrax FRET detection would be carried out using an aptamer that selectively targets Anthrax Protective Antigen 63 (PA63), a protein derived from the PA83 component of anthrax toxins.

Known aptamers could be utilized. Two examples of literature-based aptamers as may be utilized include:

(SEQ ID NO: 5)
5′-ATCACTAGTG AATTCGCGGC CGCCTGCAGG TCGACCATAT-3′
(SEQ ID NO: 6)
5′-CAGACCGTAA GGGATGCCGC CTAAACACC-3′

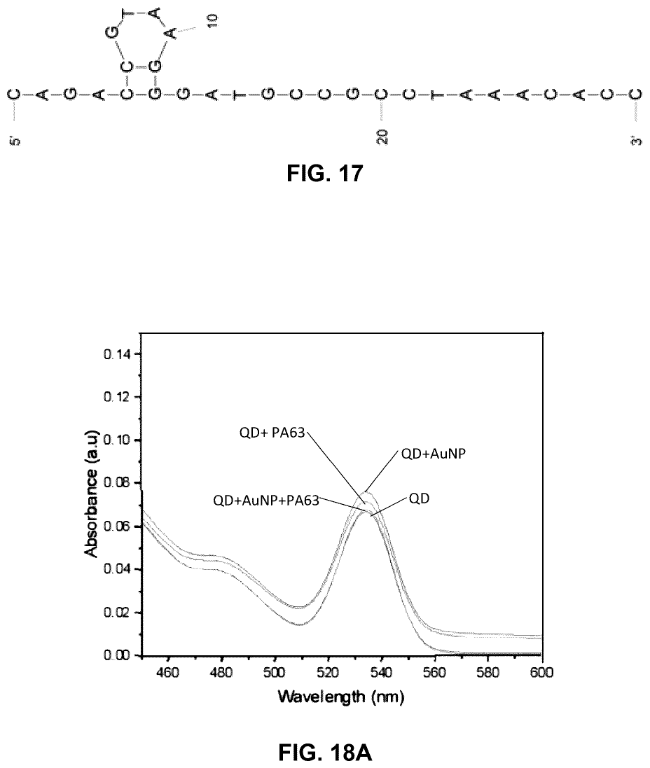

FIG. 17 illustrates the predicted secondary structure of SEQ ID NO: 6.

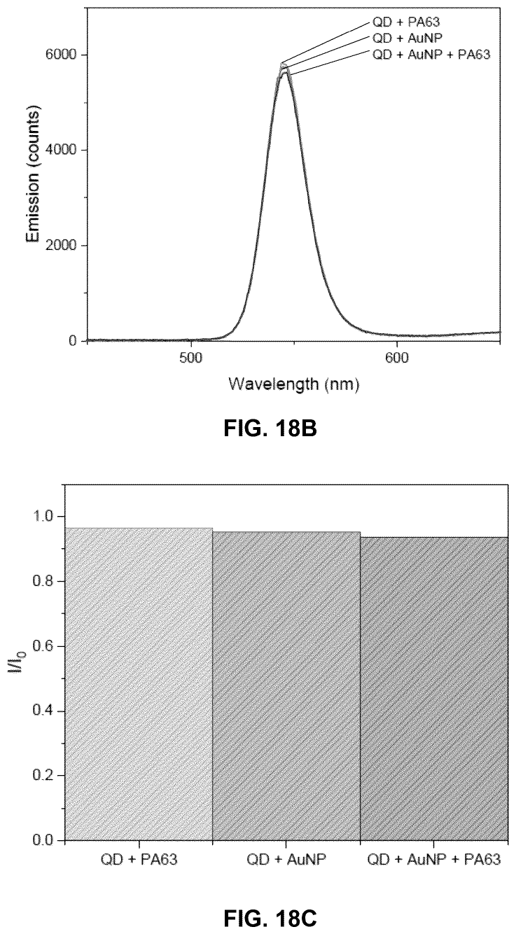

A system with the donor and acceptor of the FRET pairs unbound showed little quenching. FIG. 18A and FIG. 18B provide fluorescence spectra of the components and FIG. 18C provides a bar plot of I/I₀ of the different mixtures. As indicated, the Anthrax Protective Antigen 63 was found to have negligible quenching effects on the donor QDs in saline solution.

### Example 4

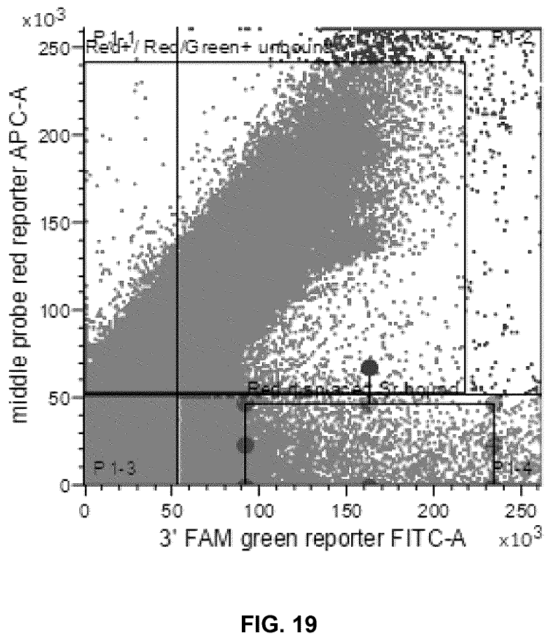

A FRET pair was also designed to target nuclear materials using an aptamer for Strontium (Sr) that was isolated using a cell sorter technique. The aptamer was also selected against common interferents (Ca, Mg, Na) for more sensitive detection. Specifically, SELEX library DNA were bound on magnetic beads and incubated with red (Alexa Fluor® 647/APC filter) and green (3′ FAM/FITC filter) fluorescent probes for 10 minutes before they were incubated with 10 nM Sr. Results are shown in FIG. 19. Sequences that bound Sr displaced the red probe and contained the green probe only were sorted out using the BD FACSMelody™. The sorted sequences were then PCR amplified for Sanger sequencing. FACS gates were determined using single stain positive controls on magnetic beads.

This written description uses examples to disclose the technology, including the best mode, and also to enable any person skilled in the art to practice the technology, including making and using any devices or systems and performing any incorporated methods. The patentable scope of the technology is defined by the claims, and may include other examples that occur to those skilled in the art. Such other examples are intended to be within the scope of the claims if they include structural elements that do not differ from the literal language of the claims, or if they include equivalent structural elements with insubstantial differences from the literal language of the claims.

1. A system for detecting chemical, biological and/or radiological/nuclear (CBRN) threats, the system comprising:
a microfluidic device, the microfluidic device comprising one or more inlet channels and one or more outlet channels;
a reservoir, the reservoir being in fluid communication with the microfluidic device; and
a sensing solution, the sensing solution comprising a sensor for the detection of one or more chemical, biological and/or radiological/nuclear (CBRN) threats, the sensor comprising:
a nanoparticle; and
a first energy transfer pair comprising a first donor, a first acceptor, and a first linking agent linking the first donor and the first acceptor, the linking agent comprising a first polynucleic acid, the first polynucleic acid comprising a first specific binding ligand for a first CBRN threat, wherein the nanoparticle is the first donor or the first acceptor.

2. The system of claim 1, wherein the nanoparticle is the first acceptor.

3. The system of claim 2, wherein the nanoparticle comprises a plasmonic metal.

4. The system of claim 3, wherein the nanoparticle comprises a noble metal.

5. The system of claim 2, wherein the first donor is selected from the group consisting of quantum dots, organic molecules, and combinations thereof.

6. The system of claim 1, wherein the nanoparticle is the first donor.

7. The system of claim 6, wherein the nanoparticle is a quantum dot.

8. The system of claim 6, wherein the first acceptor is selected from the group consisting of plasmonic metal particles, quantum dots, organic molecules, and combinations thereof.

9. The system of claim 1, wherein the first polynucleic acid comprises a first aptamer that includes the first specific binding ligand.

10. The system of claim 1, wherein the sensor comprises multiple instances of the first energy transfer pair.

11. The system of claim 1, further comprising one or more additional energy transfer pairs, each of the one or more additional energy transfer pairs being linked with a linking agent that includes a polynucleic acid and a specific binding agent for an additional CBRN threat.

12. A system for detecting chemical, biological and/or radiological/nuclear (CBRN) threats, the system comprising:
a microfluidic device, the microfluidic device comprising two or more inlet channels and one or more outlet channels, the two or more inlet channels comprising a sensing solution and an analyte solution, the sensing solution comprising a sensor for the detection of one or more chemical, biological and/or radiological/nuclear (CBRN) threats; and
a reservoir, the reservoir being in fluid communication with the microfluidic device.

13. The system of claim 12, wherein the microfluidic device has a temperature gradient across at least a portion of the microfluidic device.

14. The system of claim 12, wherein at least one channel of the microfluidic device is non-linear.

15. The system of claim 12, wherein the system further comprises a spectrophotometric device configured to obtain spectrophotometric measurements of a mixture of the sensing solution and the analyte solution.

16. A system for detecting chemical, biological and/or radiological/nuclear (CBRN) threats, the system comprising:
a mobile analysis device, the mobile analysis device comprising:
a microfluidic device, the microfluidic device comprising a sensor for the detection of one or more chemical, biological and/or
radiological/nuclear (CBRN) threats; and
a reservoir, the reservoir being in fluid communication with the microfluidic device.

17. A method for detecting chemical, biological and/or radiological/nuclear (CBRN) threats, the method comprising:
contacting a sensing solution with an analyte solution in a microfluidic device, the sensing solution comprising a sensor, the sensor comprising a nanoparticle; a first energy transfer pair comprising a first donor, a first acceptor, and a first linking agent linking the first donor and the first acceptor, the first linking agent comprising a first polynucleic acid that includes a first specific binding ligand for a first CBRN threat; and
examining the sensor for an optically detectable signal after contact of the sensing solution and the analyte solution.

18. The method of claim 17, wherein the method further comprises directing the sensing solution through a first inlet channel of the microfluidic device and directing the analyte solution through a second inlet channel of the microfluidic device.

19. The method of claim 17, wherein the optically detectable signal comprises quenching or increase of a first acceptor signal.

20. The method of claim 19, wherein the optically detectable signal is in the visible spectrum.


SEQUENCE LISTING (6 sequences)

SEQ ID NO: 1 - DNA, 72 bp, synthetic construct
  source 1..72: mol_type = other DNA
atccgtcacacctgctctcgatgagacaagaggaacacggcacaattgatttaatggtgttggctcccgtat

SEQ ID NO: 2 - skipped

SEQ ID NO: 3 - DNA, 21 bp, synthetic construct
  source 1..21: mol_type = other DNA
atttagtagcgggatctgtca

SEQ ID NO: 4 - DNA, 40 bp, synthetic construct
  source 1..40: mol_type = other DNA
aaccggcgtttagtgcctttagtcggtttagtgcctaccg

SEQ ID NO: 5 - DNA, 40 bp, synthetic construct
  source 1..40: mol_type = other DNA
atcactagtgaattcgcggccgcctgcaggtcgaccatat

SEQ ID NO: 6 - DNA, 29 bp, synthetic construct
  source 1..29: mol_type = other DNA
cagaccgtaagggatgccgcctaaacacc